(19) United States
(12) Patent Application Publication
(10) Pub. No.: US 2018/0142298 A1
(43) Pub. Date: May 24, 2018

(54) SYSTEMS AND METHODS TO PREDICT AUTISM BEFORE ONSET OF BEHAVIORAL SYMPTOMS AND/OR TO DIAGNOSE AUTISM

(71) Applicant: Bioscreening & Diagnostics LLC, Detroit, MI (US)

(72) Inventor: Ray Bahado-Singh, Grosse Pointe Shores, MI (US)

(73) Assignee: Bioscreening & Diagnostics LLC, Detroit, MI (US)

(21) Appl. No.: 15/573,726

(22) PCT Filed: May 5, 2016
(86) PCT No.: PCT/US16/30913

§ 371 Date: Nov. 13, 2017

Related U.S. Application Data
(60) Provisional application No. 62/161,630, filed on May 14, 2015.

(51) Int. Cl. C12Q 1/6883; A61B 5/16

(52) U.S. Cl. CPC C12Q 1/6883; C12Q 2600/154; C12Q 2600/118; A61B 5/16

(57) ABSTRACT

Systems and methods to predict or diagnose autism are described. Using markers, the systems and methods can predict or diagnose autism based on significant differences in methylation of cytosine bases in many loci throughout the genome. Therapeutic interventions can then be initiated at an earlier time in development, decreasing severity of the disorder.

### FIELD OF THE DISCLOSURE

The current disclosure provides systems and methods to predict autism before the onset of behavioral symptoms and/or to diagnose autism. Therapeutic interventions can then be initiated at an earlier time in development, decreasing severity of the disorder. Among other markers, the systems and methods can predict or diagnose autism based on significant differences in methylation of cytosine bases in many loci throughout the genome.

### BACKGROUND OF THE DISCLOSURE

Autism is defined as a neurodevelopmental disorder, characterized by repetitive behaviors, social withdrawal, and communication deficits. The disease has variable cognitive manifestations, ranging non-verbal individuals with cognitive deficits to those with an above-average IQ and social impairments. Population reports from developed countries show consistent, secular increases in autism prevalence. The prevalence of autism in the United States and other countries has increased since the 1970s, and particularly since the late 1990s. Overall, there is tremendous public, clinical, and scientific interest in the etiology of this disorder, its pathophysiology, and ultimately in development of targeted therapies.

### SUMMARY OF THE DISCLOSURE

The present disclosure provides systems and methods to predict autism in a subject before the development of behavioral symptoms. Additionally, systems and methods to diagnose autism are described. The systems and methods include predictive and/or diagnostic kits.

The systems and methods predict or diagnose autism based on significant differences in methylation of cytosine bases in many loci throughout the genome. The systems and methods can predict or diagnose autism in subjects of all ages including embryos, fetuses, newborns, infants, children, adolescents, and adults.

### DETAILED DESCRIPTION

Autism is defined as a neurodevelopmental disorder, characterized by repetitive behaviors, social withdrawal, and communication deficits. The prevalence of autism in the United States and other countries has increased since the 1970s, and particularly since the late 1990s. Overall, there is tremendous public, clinical, and scientific interest in the etiology of this disorder, its pathophysiology and ultimately in development of targeted therapies.

“Autism” refers to a neurodevelopmental disorder characterized by impaired social interaction, impaired verbal and non-verbal communication, and restricted and repetitive behavior. Autism includes autism spectrum disorders and/or autism spectrum disorder as defined by the Diagnostic and Statistical Manual of Mental Disorders (DSM)-IV and DSM-V, respectively.

In 1994, the American Psychiatric Association included several subtypes of autism spectrum disorders in the DSM-IV: autistic disorder, Asperger syndrome, Rett disorder, childhood disintegrative disorder (CDD), and pervasive developmental disorder not otherwise specified (PDD-NOS). (Association AP. Diagnostic and Statistical Manual of Mental Disorders. 4th ed. Washington, DC. 1994.). However, with the DSM-V in 2013, a single diagnosis, “autism spectrum disorder” has replaced previous subtypes (e.g. autistic disorder, Asperger disorder, etc.) and the clinical heterogeneity is indicated with specifiers for severity and associated conditions (e.g., intellectual impairment, language impairment).

The present disclosure provides systems and methods to predict autism in a subject before the development of behavioral symptoms or to diagnose autism following the onset of behavioral symptoms. The systems and methods include predictive or diagnostic kits.

Diagnosis of autism traditionally includes a complete history, physical examination, neurologic examination, and direct assessment of the subject's social, language, and cognitive development. If possible, sufficient time should be set aside for standardized parent interviews regarding current concerns and behavioral history, as well as structured observation of social and communicative behavior and play.

The diagnosis of autism currently is made clinically, based upon the history, examinations, and observations of behavior. It should be suspected in subjects who have abnormalities in social communication and interaction, as well as restricted, repetitive patterns of behavior, interests, and activities. Accurate and appropriate diagnosis previously required a clinician experienced in the diagnosis and treatment of autism. Reliance was placed on clinical judgment, aided by guides to diagnosis. At a minimum, the diagnostic evaluation included documentation of whether the subject's symptoms met the following DSM-V criteria for autism:
1. Persistent deficits in social communication and social interaction in multiple settings; demonstrated by deficits in all three of the following (either currently or by history):
   Social-emotional reciprocity (e.g., failure of back-and-forth conversation; and reduced sharing of interests and emotions);
   Nonverbal communicative behaviors used for social interaction (e.g., poorly integrated verbal and nonverbal communication; abnormal eye contact or body language; and poor understanding of gestures); and
   Developing, maintaining, and understanding relationships (e.g., difficulty adjusting behavior to social setting; difficulty making friends; and lack of interest in peers).
2. Restricted, repetitive patterns of behavior, interests, or activities; demonstrated by two or more of the following (either currently or by history):
   Stereotyped or repetitive movements, use of objects, or speech (e.g., stereotypes; echolalia; ordering toys; etc.);
   Insistence on sameness, unwavering adherence to routines, or ritualized patterns of behavior (verbal or nonverbal);
   Highly restricted, fixated interests that are abnormal in strength or focus (e.g., preoccupation with certain objects; perseverative interests); and
   Increased or decreased response to sensory input or unusual interest in sensory aspects of the environment (e.g., adverse response to particular sounds; apparent indifference to temperature; excessive touching/smelling of objects).
3. The symptoms must be present in the early developmental period. However, they may become apparent only after social demands exceed limited capacity. In later life, symptoms may be masked by learned strategies.
4. Symptoms together limit and impair everyday functioning.
5. The symptoms are not better explained by intellectual disability or global developmental delay

The DSM-V recommends that clinicians specify the severity level of autism, recognizing that severity may vary with context and over time. According to the DSM-V, severity should be assessed separately for social communication/interaction and repetitive/restricted behavior, and be determined by levels (Level I, II, III) depending on the support requirement.

In assessing DSM-V criteria, diagnostic evaluations should include the use of a diagnostic instrument with at least moderate sensitivity and high specificity for autism. The American Academy of Child and Adolescent Psychiatry, the American Academy of Neurology, and the American Academy of Pediatrics have recommended several such diagnostic instruments. For example, The Childhood Autism Rating Scale (CARS) and the Autism Diagnostic Observation Schedule (ADOS)-Generic (ADOS-2) can be used to measure symptoms using direct observation. For a parent of a subject, the Autism Behavior Checklist (ABC), Gilliam Autism Rating Scale, 2^{nd} edition (GARS-2), and the Autism Diagnostic Interview-Revised (ADI-R) systems can be used.

The ADOS can be used for diagnosis of autism in research studies and in clinical settings (Falkmer, et al., European J. Child and Adolescent Psychiatry 2013; 22:329-40; Lord et al., J. Autism Dev. Disorders 2000: 30:205-23.). The second edition (ADOS-2) was published in 2012. It is available for use with subjects aged one year through adulthood. The ADOS is meant to be used as one facet of an evaluation, with results to be considered in conjunction with other clinical information and the examiner's clinical expertise. The ADOS is a semi-structured assessment of social interaction, play, communication, and imaginative use of materials. It provides scores for social interaction and communication and an overall score. There are four modules based upon the child's expressive language abilities, which take 40 to 60 minutes to administer. Substantial training is required for administration and scoring. In a systematic review, the average sensitivity and specificity of the first edition of the generic ADOS were 87 and 78 percent, respectively, for autism (Falkmer, et al., European J. Child and Adolescent Psychiatry 2013; 22:329-40).

The ADOS-Toddler Module is a standardized research tool for use in children aged 12 to 30 months (or until phrase speech is acquired). It targets communication, reciprocal social interaction, emerging object use, and play skills. The Toddler Module classifies children as autistic or non-spectrum. Similar to the ADOS-2, results must be considered in conjunction with other clinical information and the examiner's clinical expertise.

The Autism Behavior Checklist (ABC) is a list of 57 questions to be completed by a parent or teacher (Krug, et al., J. Child Psychology and Psychiatry and Allied Disciplines 1980;21:221-9). The questions are divided into five categories: sensory, relating, body and object use, language, and social and self-help. It was designed primarily to identify children with autism from a population of school-age children with severe disabilities. However, it has been used with children as young as three years. The reported sensitivity and specificity of the ABC in referral samples range from 38 to 58 percent and 76 to 97 percent, respectively (Johnson, et al., Pediatr. 2007;120: 1183-215).

The third version of Gilliam Autism Rating Scale (GARS-3) was published in 2013 and is based on the DSM-V. The GARS-3 includes 56 clearly stated items describing the characteristic behaviors of persons with autism. The items are grouped into six subscales: Restrictive/Repetitive Behaviors, Social Interaction, Social Communication, Emotional Responses, Cognitive Style, and Maladaptive Speech. Testing time ranges from five to ten minutes. Internal consistency (content sampling) reliability coefficients for the subscales exceed 85% and the Autism Indexes exceed 93%. Binary classification studies indicate that the GARS-3 is able to accurately discriminate children with autism from children without autism (i.e., sensitivity=97%, specificity=97%, ROC/AUC=93%.). However, more studies of its diagnostic accuracy are required.

The Autism Diagnostic Interview-Revised (ADI-R) is a two- to three-hour clinical interview that probes for autistic symptoms. It has excellent psychometric properties (average sensitivity of 82 percent in children under three years of age and 91 percent in children over three years of age in a systematic review). The ADI-R is typically used in research settings, often combined with the ADOS-2. However, the ADI-R is not always practical for use in clinical settings because of the time required for administration.

The CARS is a 15-item direct-observation instrument designed to facilitate the diagnosis of autism in children two years of age and older. (Falkmer, et al., European J. Child and Adolescent Psychiatry 2013; 22: 329-40). Each of the items is scored on a seven-point rating scale. The CARS is well correlated with the DSM-IV criteria and discriminates ASD from other developmental disorders better than the ABC. The CARS is intended for use by a trained clinician and takes 20 to 30 minutes to administer. In a systematic review, the average sensitivity and specificity were 82 and 80 percent, respectively, for ASD (Falkmer, et al., European J. Child and Adolescent Psychiatry 2013; 22:329-40).

The foregoing description of methods to diagnose autism indicates that currently-available methods are time-consuming and subjective. Because they are subjective, uncertainty following an evaluation or diagnosis can remain. Further, none of the described methods is able to predict autism in a subject before the onset of behavioral symptoms.

The cause of autism remains unknown, and a number of potential causes are currently being assessed. Potential causes under investigation include environmental factors; immune system perturbations (e.g. maternal immunoglobulin reactivity against fetal brain proteins; altered pro-inflammatory cytokine profiles in the brain; auto-immune disorders (e.g., autistic children have serum antibody reactivity against human cortical and cerebellar brain regions)); the use of acetaminophen; folic acid metabolism deficiencies; vitamin D deficiencies; and heavy metal (e.g., mercury) toxicity. Risk factors leading to a heightened risk of autism include gestational age at birth (e.g., <35 weeks or >42 weeks), low birth weight (e.g., <2500 grams), and gender (e.g., males have a higher prevalence of autism than females).

Autism is a highly heterogeneous and complex disorder. It has proven very difficult to elucidate single-gene factors that contribute to the disorder. Much of genetic research in autism has yielded inconsistent results that suggest a wide variety of susceptibility loci and potential candidate genes, as well as a complex myriad of gene-gene and gene-environment interactions. In light of these difficulties and inconsistencies in genetic research, some insight into the genes and pathways involved in autism may be found by gaining a better understanding of epigenetic mechanisms and how they relate to the development of autism (Mbadiwe, et al., Autism Res. Treat. 2013; 2013:826156. Epub 2013 Sep. 15.).

Epigenetics refers to heritable changes in gene expression that are not due to mutations (i.e. changes in the sequence, such as loss or gain of nucleotides, of a gene) and is an important mechanism for controlling gene function. In other words, epigenetics is a reversible regulation of gene expression by several mechanisms other than mutation. For example, epigenetic modification is the mechanism by which cells which contain identical DNA are able to activate different genes and result in the differentiation into unique tissues (e.g. heart or intestines).

One example of an epigenetic mechanism is DNA methylation. Other mechanisms include changes to the three dimensional structure of DNA, histone protein modification, micro-RNA inhibitory activity, imprinting, X-inactivation, and long-distance chromosomal interaction.

Epigenetic alterations result in part from the effects of the environment, thus the environment plays a role in the phenotype by modulating gene expression. Epigenetic processes and their interaction with environmental conditions could explain the differences in gene expression within subjects with autism despite minimal consistent evidence of specific gene mutations. (Persico, et al., Trends in Neurosciences 2006; 29:349-58). Although epigenetic mechanisms involved in autism are not fully understood, there is evidence for genome-wide methylation dysregulation in autism. (Nguyen, FASEB 2010; 24:3036-51).

Methionine synthase is an important enzyme in the folate metabolism pathway. Inhibition of methionine synthase affects methylation activity, and reduced DNA methylation interferes with normal development and proper gene silencing. Impaired phospholipid methylation leads to disruption of neuronal networks, consequently leading to attention and cognitive deficits.

The systems and methods disclosed herein predict or diagnose autism based on the methylation status of cytosine at various loci in the genome.

Cytosine is one of a group of four building blocks (i.e., nucleotides) from which DNA is constructed (i.e. cytosine (C), thiamine (T), adenine (A), and guanosine (G)). The chemical structure of cytosine is in the form of a six-sided hexagon or pyrimidine ring. Cytosine is usually paired with guanosine in a linear sequence along the single DNA strand to form CpG pairs. “CpG” refers to a cytosine-phosphate-guanosine chemical bond in which the phosphate binds the two nucleotides together. In mammals, in 70-80% of these CpG pairs the cytosine is methylated. (Chatterjee, et al., Biochemica et Biophisica Acta 2012; 1819:763-70).

The term “CpG island” refers to regions in the genome with a high concentration of CG dinucleotide pairs or CpG sites. The length of DNA occupied by the CpG island is usually 300-3000 base pairs. The CpG island is defined by various criteria including the length of recurrent CG dinucleotide pairs occupying at least 200 base pair (bp) of DNA, a CG content of the segment of at least 50%, and that the observed/expected CpG ratio is greater than 60%.

Forty percent of promoter region of mammalian genes have associated CpG islands and three quarters of these promoter-regions have high CpG concentrations. (Fatemi, et al., Nucleic Acids Res. 2005; 33:e176). In humans 70% of the promoter regions of genes have high CG content. The CG dinucleotide pairs may exist elsewhere in the gene or outside of the gene and are not known to be associated with any particular genes.

In most CpG sites scattered throughout the DNA the cytosine nucleotide is methylated. In contrast, the cytosine is more often unmethylated in CpG sites located in the CpG islands of the promoter regions of genes, suggesting a role of methylation status of cytosine in CpG Islands in gene transcriptional activity.

Methylation of cytosine refers to the enzymatic addition of a “methyl group” or single carbon atom to position #5 of the pyrimidine ring of cytosine, which leads to the conversion of cytosine to 5-methyl-cytosine. The methylation of cytosine can be accomplished by a family of enzymes called DNA methyltransferases (DNMT's). The 5-methyl-cytosine, when formed, is prone to mutation or the chemical transformation of the original cytosine to form thymine. Five-methyl-cytosines account for 1% of the nucleotide bases overall in the normal genome.

As indicated previously, the methylation status of cytosine throughout the DNA can be said to indirectly indicate the relative expression status of multiple genes throughout the genome. The methylation of cytosine nucleotides within a gene, particularly in the promoter region of the gene, is known to be a mechanism of controlling overall gene activity, i.e. mRNA and protein synthesis. Classically, the methylation of cytosine is associated with inhibition of gene transcription. However, in certain genes, methylation of cytosine is known to have the reverse effect and instead promotes gene transcription.

Disclosed herein is that highly statistically significant differences exist in the percentage or level of methylation of individual cytosine nucleotides distributed throughout the genome when autistic subjects are compared to non-autistic subjects. Cytosines demonstrating methylation differences are distributed both inside and outside of CpG islands and genes. Disclosed herein are methylation markers within and outside of genes for distinguishing a subject with autism from unaffected subjects.

A collection of genes that are involved in epigenetic pathways of autism has been identified. Specifically, the GABRB3, UBE3A, and MECP23 genes have been consistently shown to be epigenetically dysregulated in autism. There is also a growing body of literature to support the epigenetic role of other genes in autism, such as the RELN gene (Flashner, et al., Neuromolecular Med. 2013; 15:339-50), which plays a role in neuronal migration and synaptogenesis; the oxytocin receptor (OXTR) gene (Gregory, et al., BMC Medicine 2009; 7:62.), which is involved in social behavior; and the engrailed-2 gene (EN-2), which is responsible for Purkinje cell maturation (James, et al., Translational Psychiatry 2013; 3:e232). Recently, methylation profiling revealed differential methylation patterns between subjects with autism and their non-autistic siblings, identifying the retinoic acid-related orphan receptor alpha (RORA) gene as an epigenetically dysregulated gene in autism (Nguyen, et al., FASEB 2010; 24:3036-51).

Various embodiments relate to the measurements of cytosine methylation and its use in predicting and/or diagnosing autism. In addition, assaying the concentrations of mRNA and/or proteins that are the products of the differentially expressed genes (due to differences in cytosine methylation) in order to predict and/or diagnose autism are disclosed. Additionally, in various embodiments, systems and methods disclosed herein use statistical algorithms to predict or diagnose autism based on methylation levels at informative cytosine loci.

In various embodiments, autism is predicted and/or diagnosed in a subject by assaying the methylation of a genetic loci and/or the up- or down-regulation of cDNA, mRNA, and/or proteins associated with the gene's expression and/or methylation status. Particular markers are selected from BCOR (BCL6 co-repressor; chromosome X), C8orf75 (long intergenic non-protein coding RNA 589; chromosome 8), CLCN1 (chloride channel, voltage-sensitive chloride channel 1), CLCN4 (chloride channel, voltage-sensitive 4; chromosome X), DIP2C (DIP2 disco-interacting protein 2 homolog 0; chromosome 10), GPM6B (glycoprotein M613, chromosome X), ITGAX (integrin, alpha X (complement component 3 receptor 4 subunit); chromosome 16), LOC284412 (chromosome 2), LOC285375 (long intergenic non-protein coding RNA 620; chromosome 3), MAMLD1 (mastermind-like domain containing 1; chromosome X), MGC16121 (MIR503 host gene; chromosome X), NDUFA10 (NADH dehydrogenase (ubiquinone) 1 alpha subcomplex, 10; chromosome 2), PDE9A (phosphodiesterase 9A; chromosome 21), PPAPDC1A (phosphatidic acid phosphatase type 2 domain containing 1A; chromosome 10), PTPRN2 (protein tyrosine phosphatase, receptor type, N polypeptide 2; chromosome 7), RAP1GAP2 (RAP1 GTPase activating protein 2; chromosome 17), RPS4Y2 (ribosomal protein S4, Y-linked 2; chromosome Y), TUBA3D (tubulin, alpha 3d; chromosome 2), and UBTD1 (ubiquitin domain containing 1; chromosome 10).

In particular embodiments, the systems and methods predict or diagnose autism by assaying a sample obtained from a subject for the methylation status, up- or down-regulation of two or more; three or more; four or more; five or more; six or more; seven or more; eight or more; nine or more or ten or more markers associated with autism disclosed herein. In further embodiments, the systems and methods predict or diagnose autism by assaying a sample obtained from a subject for the methylation status, up- or down-regulation of two, three, four, five, six, seven, eight, nine, or ten markers associated with autism disclosed herein.

In particular embodiments, the markers include (hereafter referred to by gene abbreviations for brevity) BCOR, PTPRN2, TUBA3D, PDE9A, and LOC284412. In particular embodiments, the markers include PTPRN2, TUBA3D, PDE9A, and LOC284412. In particular embodiments, the markers include BCOR, TUBA3D, PDE9A, and LOC284412. In particular embodiments, the markers include BCOR, PTPRN2, PDE9A, and LOC284412. In particular embodiments, the markers include BCOR, PTPRN2, TUBA3D, and LOC284412. In particular embodiments, the markers include BCOR, PTPRN2, TUBA3D, and PDE9A. In particular embodiments, the markers include TUBA3D, PDE9A, and LOC284412; PTPRN2, PDE9A, and LOC284412; PTPRN2, TUBA3D, and LOC284412; PTPRN2, TUBA3D, and PDE9A; BCOR, PDE9A, and LOC284412; BCOR, TUBA3D, and LOC284412; BCOR, TUBA3D, and PDE9A; BCOR, PTPRN2, and LOC284412; BCOR, PTPRN2, and PDE9A; or BCOR, PTPRN2, and TUBA3D.

In particular embodiments, the markers include GPM6B, NDUFA10, PDE9A, and LOC284412. In particular embodiments, the markers include GPM6B, NDUFA10, and PDE9A. In particular embodiments, the markers include GPM6B, NDUFA10, and LOC284412. In particular embodiments, the markers include GPM6B, PDE9A, and LOC284412. In particular embodiments, the markers include NDUFA10, PDE9A, and LOC284412.

In particular embodiments, the markers include BCOR, UBTD1, LOC285375, RPS4Y2, PPAPDC1A, ITGAX, DIP2C, MGC16121, PTPRN2, and CLCN4. In particular embodiments, the markers include UBTD1, LOC285375, RPS4Y2, PPAPDC1A, ITGAX, DIP2C, MGC16121, PTPRN2, and CLCN4. In particular embodiments, the markers include BCOR, LOC285375, RPS4Y2, PPAPDC1A, ITGAX, DIP2C, MGC16121, PTPRN2, and CLCN4. In particular embodiments, the markers include BCOR, UBTD1, RPS4Y2, PPAPDC1A, ITGAX, DIP2C, MGC16121, PTPRN2, and CLCN4. In particular embodiments, the markers include BCOR, UBTD1, LOC285375, PPAPDC1A, ITGAX, DIP2C, MGC16121, PTPRN2, and CLCN4. In particular embodiments, the markers include BCOR, UBTD1, LOC285375, RPS4Y2, ITGAX, DIP2C, MGC16121, PTPRN2, and CLCN4. In particular embodiments, the markers include BCOR, UBTD1, LOC285375, RPS4Y2, PPAPDC1A, DIP2C, MGC16121, PTPRN2, and CLCN4. In particular embodiments, the markers include BCOR, UBTD1, LOC285375, RPS4Y2, PPAPDC1A, ITGAX, MGC16121, PTPRN2, and CLCN4. In particular embodiments, the markers include BCOR, UBTD1, LOC285375, RPS4Y2, PPAPDC1A, ITGAX, DIP2C, PTPRN2, and CLCN4. In particular embodiments, the markers include BCOR, UBTD1, LOC285375, RPS4Y2, PPAPDC1A, ITGAX, DIP2C, MGC16121, and CLCN4. In particular embodiments, the markers include BCOR, UBTD1, LOC285375, RPS4Y2, PPAPDC1A, ITGAX, DIP2C, MGC16121, and PTPRN2. In particular embodiments, the markers include LOC285375, RPS4Y2, PPAPDC1A, ITGAX, DIP2C, MGC16121, PTPRN2, and CLCN4; UBTD1, RPS4Y2, PPAPDC1A, ITGAX, DIP2C, MGC16121, PTPRN2, and CLCN4; UBTD1, LOC285375, PPAPDC1A, ITGAX, DIP2C, MGC16121, PTPRN2, and CLCN4; UBTD1, LOC285375, RPS4Y2, ITGAX, DIP2C, MGC16121, PTPRN2, and CLCN4; UBTD1, LOC285375, RPS4Y2, PPAPDC1A, DIP2C, MGC16121, PTPRN2, and CLCN4; UBTD1, LOC285375, RPS4Y2, PPAPDC1A, ITGAX, MGC16121, PTPRN2, and CLCN4; UBTD1, LOC285375, RPS4Y2, PPAPDC1A, ITGAX, DIP2C, PTPRN2, and CLCN4; UBTD1, LOC285375, RPS4Y2, PPAPDC1A, ITGAX, DIP2C, MGC16121, and CLCN4; UBTD1, LOC285375, RPS4Y2, PPAPDC1A, ITGAX, DIP2C, MGC16121, and PTPRN2; BCOR, RPS4Y2, PPAPDC1A, ITGAX, DIP2C, MGC16121, PTPRN2, and CLCN4; BCOR, LOC285375, PPAPDC1A, ITGAX, DIP2C, MGC16121, PTPRN2, and CLCN4; BCOR, LOC285375, RPS4Y2, ITGAX, DIP2C, MGC16121, PTPRN2, and CLCN4; BCOR, LOC285375, RPS4Y2, PPAPDC1A, DIP2C, MGC16121, PTPRN2, and CLCN4; BCOR, LOC285375, RPS4Y2, PPAPDC1A, ITGAX, MGC16121, PTPRN2, and CLCN4; BCOR, LOC285375, RPS4Y2, PPAPDC1A, ITGAX, DIP2C, PTPRN2, and CLCN4; BCOR, LOC285375, RPS4Y2, PPAPDC1A, ITGAX, DIP2C, MGC16121, and CLCN4; BCOR, LOC285375, RPS4Y2, PPAPDC1A, ITGAX, DIP2C, MGC16121, and PTPRN2; BCOR, UBTD1, PPAPDC1A, ITGAX, DIP2C, MGC16121, PTPRN2, and CLCN4; BCOR, UBTD1, RPS4Y2, ITGAX, DIP2C, MGC16121, PTPRN2, and CLCN4; BCOR, UBTD1, RPS4Y2, PPAPDC1A, DIP2C, MGC16121, PTPRN2, and CLCN4; BCOR, UBTD1, RPS4Y2, PPAPDC1A, ITGAX, MGC16121, PTPRN2, and CLCN4; BCOR, UBTD1, RPS4Y2, PPAPDC1A, ITGAX, DIP2C, PTPRN2, and CLCN4; BCOR, UBTD1, RPS4Y2, PPAPDC1A, ITGAX, DIP2C, MGC16121, and CLCN4; BCOR, UBTD1, RPS4Y2, PPAPDC1A, ITGAX, DIP2C, MGC16121, and PTPRN2; BCOR, UBTD1, LOC285375, ITGAX, DIP2C, MGC16121, PTPRN2, and CLCN4; BCOR, UBTD1, LOC285375, PPAPDC1A, DIP2C, MGC16121, PTPRN2, and CLCN4; BCOR, UBTD1, LOC285375, PPAPDC1A, ITGAX, MGC16121, PTPRN2, and CLCN4; BCOR, UBTD1, LOC285375, PPAPDC1A, ITGAX, DIP2C, PTPRN2, and CLCN4; BCOR, UBTD1, LOC285375, PPAPDC1A, ITGAX, DIP2C, MGC16121, and CLCN4; BCOR, UBTD1, LOC285375, PPAPDC1A, ITGAX, DIP2C, MGC16121, and PTPRN2; BCOR, UBTD1, LOC285375, RPS4Y2, DIP2C, MGC16121, PTPRN2, and CLCN4; BCOR, UBTD1, LOC285375, RPS4Y2, ITGAX, MGC16121, PTPRN2, and CLCN4; BCOR, UBTD1, LOC285375, RPS4Y2, ITGAX, DIP2C, PTPRN2, and CLCN4; BCOR, UBTD1, LOC285375, RPS4Y2, ITGAX, DIP2C, MGC16121, and CLCN4; BCOR, UBTD1, LOC285375, RPS4Y2, ITGAX, DIP2C, MGC16121, and PTPRN2; BCOR, UBTD1, LOC285375, RPS4Y2, PPAPDC1A, MGC16121, PTPRN2, and CLCN4; BCOR, UBTD1, LOC285375, RPS4Y2, PPAPDC1A, DIP2C, PTPRN2, and CLCN4; BCOR, UBTD1, LOC285375, RPS4Y2, PPAPDC1A, DIP2C, MGC16121, and CLCN4; BCOR, UBTD1, LOC285375, RPS4Y2, PPAPDC1A, DIP2C, MGC16121, and PTPRN2; BCOR, UBTD1, LOC285375, RPS4Y2, PPAPDC1A, ITGAX, PTPRN2, and CLCN4; BCOR, UBTD1, LOC285375, RPS4Y2, PPAPDC1A, ITGAX, MGC16121, and CLCN4; BCOR, UBTD1, LOC285375, RPS4Y2, PPAPDC1A, ITGAX, MGC16121, and PTPRN2; BCOR, UBTD1, LOC285375, RPS4Y2, PPAPDC1A, ITGAX, DIP2C, and CLCN4; BCOR, UBTD1, LOC285375, RPS4Y2, PPAPDC1A, ITGAX, DIP2C, and PTPRN2; or BCOR, UBTD1, LOC285375, RPS4Y2, PPAPDC1A, ITGAX, DIP2C, and MGC16121. In particular embodiments, the markers include RPS4Y2, PPAPDC1A, ITGAX, DIP2C, MGC16121, PTPRN2, and CLCN4; LOC285375, PPAPDC1A, ITGAX, DIP2C, MGC16121, PTPRN2, and CLCN4; LOC285375, RPS4Y2, ITGAX, DIP2C, MGC16121, PTPRN2, and CLCN4; LOC285375, RPS4Y2, PPAPDC1A, DIP2C, MGC16121, PTPRN2, and CLCN4; LOC285375, RPS4Y2, PPAPDC1A, ITGAX, MGC16121, PTPRN2, and CLCN4; LOC285375, RPS4Y2, PPAPDC1A, ITGAX, DIP2C, PTPRN2, and CLCN4; LOC285375, RPS4Y2, PPAPDC1A, ITGAX, DIP2C, MGC16121, and CLCN4; LOC285375, RPS4Y2, PPAPDC1A, ITGAX, DIP2C, MGC16121, PTPRN2; UBTD1,PPAPDC1A, ITGAX, DIP2C, MGC16121, PTPRN2, and CLCN4; UBTD1, RPS4Y2, ITGAX, DIP2C, MGC16121, PTPRN2, and CLCN4; UBTD1, RPS4Y2, PPAPDC1A, DIP2C, MGC16121, PTPRN2, and CLCN4; UBTD1, RPS4Y2, PPAPDC1A, ITGAX, MGC16121, PTPRN2, and CLCN4; UBTD1, RPS4Y2, PPAPDC1A, ITGAX, DIP2C, PTPRN2, and CLCN4; UBTD1, RPS4Y2, PPAPDC1A, ITGAX, DIP2C, MGC16121, and CLCN4; UBTD1, RPS4Y2, PPAPDC1A, ITGAX, DIP2C, MGC16121, PTPRN2; UBTD1, LOC285375, ITGAX, DIP2C, MGC16121, PTPRN2, and CLCN4; UBTD1, LOC285375, PPAPDC1A, DIP2C, MGC16121, PTPRN2, and CLCN4; UBTD1, LOC285375, PPAPDC1A, ITGAX, MGC16121, PTPRN2, and CLCN4; UBTD1, LOC285375, PPAPDC1A, ITGAX, DIP2C, PTPRN2, and CLCN4; UBTD1, LOC285375, PPAPDC1A, ITGAX, DIP2C, MGC16121, and CLCN4; UBTD1, LOC285375, PPAPDC1A, ITGAX, DIP2C, MGC16121, PTPRN2; UBTD1, LOC285375, RPS4Y2, DIP2C, MGC16121, PTPRN2, and CLCN4; UBTD1, LOC285375, RPS4Y2, ITGAX, MGC16121, PTPRN2, and CLCN4; UBTD1, LOC285375, RPS4Y2, ITGAX, DIP2C, PTPRN2, and CLCN4; UBTD1, LOC285375, RPS4Y2, ITGAX, DIP2C, MGC16121,and CLCN4; UBTD1, LOC285375, RPS4Y2, ITGAX, DIP2C, MGC16121, PTPRN2; UBTD1, LOC285375, RPS4Y2, PPAPDC1A, MGC16121, PTPRN2, and CLCN4; UBTD1, LOC285375, RPS4Y2, PPAPDC1A, DIP2C, PTPRN2, and CLCN4; UBTD1, LOC285375, RPS4Y2, PPAPDC1A, DIP2C, MGC16121, and CLCN4; UBTD1, LOC285375, RPS4Y2, PPAPDC1A, DIP2C, MGC16121, PTPRN2; UBTD1, LOC285375, RPS4Y2, PPAPDC1A, ITGAX, PTPRN2, and CLCN4; UBTD1, LOC285375, RPS4Y2, PPAPDC1A, ITGAX, MGC16121, and CLCN4; UBTD1, LOC285375, RPS4Y2, PPAPDC1A, ITGAX, MGC16121, PTPRN2; UBTD1, LOC285375, RPS4Y2, PPAPDC1A, ITGAX, DIP2C, and CLCN4; UBTD1, LOC285375, RPS4Y2, PPAPDC1A, ITGAX, DIP2C, PTPRN2; UBTD1, LOC285375, RPS4Y2, PPAPDC1A, ITGAX, DIP2C, MGC16121,; BCOR, PPAPDC1A, ITGAX, DIP2C, MGC16121, PTPRN2, and CLCN4; BCOR, RPS4Y2,ITGAX, DIP2C, MGC16121, PTPRN2, and CLCN4; BCOR, RPS4Y2, PPAPDC1A, DIP2C, MGC16121, PTPRN2, and CLCN4; BCOR, RPS4Y2, PPAPDC1A, ITGAX, MGC16121, PTPRN2, and CLCN4; BCOR, RPS4Y2, PPAPDC1A, ITGAX, DIP2C, PTPRN2, and CLCN4; BCOR, RPS4Y2, PPAPDC1A, ITGAX, DIP2C, MGC16121, and CLCN4; BCOR, RPS4Y2, PPAPDC1A, ITGAX, DIP2C, MGC16121, PTPRN2; BCOR, LOC285375, ITGAX, DIP2C, MGC16121, PTPRN2, and CLCN4; BCOR, LOC285375, PPAPDC1A, DIP2C, MGC16121, PTPRN2, and CLCN4; BCOR, LOC285375, PPAPDC1A, ITGAX, MGC16121, PTPRN2, and CLCN4; BCOR, LOC285375, PPAPDC1A, ITGAX, DIP2C, PTPRN2, and CLCN4; BCOR, LOC285375, PPAPDC1A, ITGAX, DIP2C, MGC16121, and CLCN4; BCOR, LOC285375, PPAPDC1A, ITGAX, DIP2C, MGC16121, PTPRN2; BCOR, LOC285375, RPS4Y2, DIP2C, MGC16121, PTPRN2, and CLCN4; BCOR, LOC285375, RPS4Y2, ITGAX, MGC16121, PTPRN2, and CLCN4; BCOR, LOC285375, RPS4Y2, ITGAX, DIP2C, PTPRN2, and CLCN4; BCOR, LOC285375, RPS4Y2, ITGAX, DIP2C, MGC16121, and CLCN4; BCOR, LOC285375, RPS4Y2, ITGAX, DIP2C, MGC16121, PTPRN2; BCOR, LOC285375, RPS4Y2, PPAPDC1A, MGC16121, PTPRN2, and CLCN4; BCOR, LOC285375, RPS4Y2, PPAPDC1A, DIP2C, PTPRN2, and CLCN4; BCOR, LOC285375, RPS4Y2, PPAPDC1A, DIP2C, MGC16121, and CLCN4; BCOR, LOC285375, RPS4Y2, PPAPDC1A, DIP2C, MGC16121, PTPRN2; BCOR, LOC285375, RPS4Y2, PPAPDC1A, ITGAX, PTPRN2, and CLCN4; BCOR, LOC285375, RPS4Y2, PPAPDC1A, ITGAX, MGC16121, and CLCN4; BCOR, LOC285375, RPS4Y2, PPAPDC1A, ITGAX, MGC16121, PTPRN2; BCOR, LOC285375, RPS4Y2, PPAPDC1A, ITGAX, DIP2C, and CLCN4; BCOR, LOC285375, RPS4Y2, PPAPDC1A, ITGAX, DIP2C, PTPRN2; BCOR, LOC285375, RPS4Y2, PPAPDC1A, ITGAX, DIP2C, MGC16121,; BCOR, UBTD1, ITGAX, DIP2C, MGC16121, PTPRN2, and CLCN4; BCOR, UBTD1, PPAPDC1A, DIP2C, MGC16121, PTPRN2, and CLCN4; BCOR, UBTD1, PPAPDC1A, ITGAX, MGC16121, PTPRN2, and CLCN4; BCOR, UBTD1, PPAPDC1A, ITGAX, DIP2C, PTPRN2, and CLCN4; BCOR, UBTD1, PPAPDC1A, ITGAX, DIP2C, MGC16121, and CLCN4; BCOR, UBTD1, PPAPDC1A, ITGAX, DIP2C, MGC16121, PTPRN2; BCOR, UBTD1, RPS4Y2, DIP2C, MGC16121, PTPRN2, and CLCN4; BCOR, UBTD1, RPS4Y2, ITGAX, MGC16121, PTPRN2, and CLCN4; BCOR, UBTD1, RPS4Y2, ITGAX, DIP2C, PTPRN2, and CLCN4; BCOR, UBTD1, RPS4Y2, ITGAX, DIP2C, MGC16121, and CLCN4; BCOR, UBTD1, RPS4Y2, ITGAX, DIP2C, MGC16121, PTPRN2; BCOR, UBTD1, RPS4Y2, PPAPDC1A, MGC16121, PTPRN2, and CLCN4; BCOR, UBTD1, RPS4Y2, PPAPDC1A, DIP2C, PTPRN2, and CLCN4; BCOR, UBTD1, RPS4Y2, PPAPDC1A, DIP2C, MGC16121, and CLCN4; BCOR, UBTD1, RPS4Y2, PPAPDC1A, DIP2C, MGC16121, PTPRN2; BCOR, UBTD1, RPS4Y2, PPAPDC1A, ITGAX, PTPRN2, and CLCN4; BCOR, UBTD1, RPS4Y2, PPAPDC1A, ITGAX, MGC16121, and CLCN4; BCOR, UBTD1, RPS4Y2, PPAPDC1A, ITGAX, MGC16121, PTPRN2; BCOR, UBTD1, RPS4Y2, PPAPDC1A, ITGAX, DIP2C, and CLCN4; BCOR, UBTD1, RPS4Y2, PPAPDC1A, ITGAX, DIP2C, PTPRN2; BCOR, UBTD1, RPS4Y2, PPAPDC1A, ITGAX, DIP2C, MGC16121; BCOR, UBTD1, LOC285375, DIP2C, MGC16121, PTPRN2, and CLCN4; BCOR, UBTD1, LOC285375, RPS4Y2ITGAX, MGC16121, PTPRN2, and CLCN4; BCOR, UBTD1, LOC285375, ITGAX, DIP2C, PTPRN2, and CLCN4; BCOR, UBTD1, LOC285375, ITGAX, DIP2C, MGC16121, and CLCN4; BCOR, UBTD1, LOC285375, ITGAX, DIP2C, MGC16121, PTPRN2; BCOR, UBTD1, LOC285375, PPAPDC1A, MGC16121, PTPRN2, and CLCN4; BCOR, UBTD1, LOC285375, PPAPDC1A, DIP2C, PTPRN2, and CLCN4; BCOR, UBTD1, LOC285375, PPAPDC1A, DIP2C, MGC16121, and CLCN4; BCOR, UBTD1, LOC285375, PPAPDC1A, DIP2C, MGC16121, PTPRN2; BCOR, UBTD1, LOC285375, PPAPDC1A, ITGAX, PTPRN2, and CLCN4; BCOR, UBTD1, LOC285375, PPAPDC1A, ITGAX, MGC16121, and CLCN4; BCOR, UBTD1, LOC285375, PPAPDC1A, ITGAX, MGC16121, PTPRN2; BCOR, UBTD1, LOC285375, PPAPDC1A, ITGAX, DIP2C, and CLCN4; BCOR, UBTD1, LOC285375, PPAPDC1A, ITGAX, DIP2C, PTPRN2; BCOR, UBTD1, LOC285375, PPAPDC1A, ITGAX, DIP2C, MGC16121, and; BCOR, UBTD1, LOC285375, RPS4Y2, MGC16121, PTPRN2, and CLCN4; BCOR, UBTD1, LOC285375, RPS4Y2, DIP2C, PTPRN2, and CLCN4; BCOR, UBTD1, LOC285375, RPS4Y2, DIP2C, MGC16121, and CLCN4; BCOR, UBTD1, LOC285375, RPS4Y2,DIP2C, MGC16121, PTPRN2; BCOR, UBTD1, LOC285375, RPS4Y2, ITGAX, PTPRN2, and CLCN4; BCOR, UBTD1, LOC285375, RPS4Y2, ITGAX, MGC16121, and CLCN4; BCOR, UBTD1, LOC285375, RPS4Y2, ITGAX, MGC16121, PTPRN2; BCOR, UBTD1, LOC285375, RPS4Y2, ITGAX, DIP2C, and CLCN4; BCOR, UBTD1, LOC285375, RPS4Y2, ITGAX, DIP2C, PTPRN2; BCOR, UBTD1, LOC285375, RPS4Y2, ITGAX, DIP2C, MGC16121; BCOR, UBTD1, LOC285375, RPS4Y2, PPAPDC1A, PTPRN2, and CLCN4; BCOR, UBTD1, LOC285375, RPS4Y2, PPAPDC1A, MGC16121, and CLCN4; BCOR, UBTD1, LOC285375, RPS4Y2, PPAPDC1A, MGC16121, PTPRN2; BCOR, UBTD1, LOC285375, RPS4Y2, PPAPDC1A, DIP2C, and CLCN4; BCOR, UBTD1, LOC285375, RPS4Y2, PPAPDC1A, DIP2C, PTPRN2; BCOR, UBTD1, LOC285375, RPS4Y2, PPAPDC1A, DIP2C, MGC16121, and; BCOR, UBTD1, LOC285375, RPS4Y2, PPAPDC1A, ITGAX, and CLCN4; BCOR, UBTD1, LOC285375, RPS4Y2, PPAPDC1A, ITGAX, PTPRN2; BCOR, UBTD1, LOC285375, RPS4Y2, PPAPDC1A, ITGAX, and MGC16121; or BCOR, UBTD1, LOC285375, RPS4Y2, PPAPDC1A, ITGAX, and DIP2C. In particular embodiments, the markers include BCOR, UBTD1, LOC285375, RPS4Y2, PPAPDC1A, and ITGAX; BCOR, UBTD1, LOC285375, RPS4Y2, PPAPDC1A, and DIP2C; BCOR, UBTD1, LOC285375, RPS4Y2, PPAPDC1A, and MGC16121; BCOR, UBTD1, LOC285375, RPS4Y2, PPAPDC1A, and PTPRN2; BCOR, UBTD1, LOC285375, RPS4Y2, PPAPDC1A, and CLCN4; BCOR, UBTD1, LOC285375, RPS4Y2, ITGAX, and DIP2C; BCOR, UBTD1, LOC285375, RPS4Y2, ITGAX, and MGC16121; BCOR, UBTD1, LOC285375, RPS4Y2, ITGAX, and PTPRN2; BCOR, UBTD1, LOC285375, RPS4Y2, ITGAX, and CLCN4; BCOR, UBTD1, LOC285375, RPS4Y2, DIP2C, and MGC16121; BCOR, UBTD1, LOC285375, RPS4Y2, DIP2C, and PTPRN2; BCOR, UBTD1, LOC285375, RPS4Y2, DIP2C, and CLCN4; BCOR, UBTD1, LOC285375, RPS4Y2, MGC16121, and PTPRN2; BCOR, UBTD1, LOC285375, RPS4Y2, MGC16121, and CLCN4; BCOR, UBTD1, LOC285375, RPS4Y2, PTPRN2, and CLCN4; BCOR, UBTD1, LOC285375, PPAPDC1A, ITGAX, and DIP2C; BCOR, UBTD1, LOC285375, PPAPDC1A, ITGAX, and MGC16121; BCOR, UBTD1, LOC285375, PPAPDC1A, ITGAX, and PTPRN2; BCOR, UBTD1, LOC285375, PPAPDC1A, ITGAX, and CLCN4; BCOR, UBTD1, LOC285375, PPAPDC1A, DIP2C, and MGC16121; BCOR, UBTD1, LOC285375, PPAPDC1A, DIP2C, and PTPRN2; BCOR, UBTD1, LOC285375, PPAPDC1A, DIP2C, and CLCN4; BCOR, UBTD1, LOC285375, PPAPDC1A, MGC16121, and PTPRN2; BCOR, UBTD1, LOC285375, PPAPDC1A, MGC16121, and CLCN4; BCOR, UBTD1, LOC285375, PPAPDC1A, PTPRN2, and CLCN4; BCOR, UBTD1, LOC285375, ITGAX, DIP2C, and MGC16121; BCOR, UBTD1, LOC285375, ITGAX, DIP2C, and PTPRN2; BCOR, UBTD1, LOC285375, ITGAX, DIP2C, and CLCN4; BCOR, UBTD1, LOC285375, ITGAX, MGC16121, and PTPRN2; BCOR, UBTD1, LOC285375, ITGAX, MGC16121, and CLCN4; BCOR, UBTD1, LOC285375, ITGAX, PTPRN2, and CLCN4; BCOR, UBTD1, LOC285375, DIP2C, MGC16121, and PTPRN2; BCOR, UBTD1, LOC285375, DIP2C, MGC16121, and CLCN4; BCOR, UBTD1, LOC285375, DIP2C, PTPRN2, and CLCN4; BCOR, UBTD1, LOC285375, MGC16121, PTPRN2, and CLCN4; BCOR, UBTD1, RPS4Y2, PPAPDC1A, ITGAX, and DIP2C; BCOR, UBTD1, RPS4Y2, PPAPDC1A, ITGAX, and MGC16121; BCOR, UBTD1, RPS4Y2, PPAPDC1A, ITGAX, and PTPRN2; BCOR, UBTD1, RPS4Y2, PPAPDC1A, ITGAX, and CLCN4; BCOR, UBTD1, RPS4Y2, PPAPDC1A, DIP2C, and MGC16121; BCOR, UBTD1, RPS4Y2, PPAPDC1A, DIP2C, and PTPRN2; BCOR, UBTD1, RPS4Y2, PPAPDC1A, DIP2C, and CLCN4; BCOR, UBTD1, RPS4Y2, PPAPDC1A, MGC16121, and PTPRN2; BCOR, UBTD1, RPS4Y2, PPAPDC1A, MGC16121, and CLCN4; BCOR, UBTD1, RPS4Y2, PPAPDC1A, PTPRN2, and CLCN4; BCOR, UBTD1, RPS4Y2, ITGAX, DIP2C, and MGC16121; BCOR, UBTD1, RPS4Y2, ITGAX, DIP2C, and PTPRN2; BCOR, UBTD1, RPS4Y2, ITGAX, DIP2C, and CLCN4; BCOR, UBTD1, RPS4Y2, ITGAX, MGC16121, and PTPRN2; BCOR, UBTD1, RPS4Y2, ITGAX, MGC16121, and CLCN4; BCOR, UBTD1, RPS4Y2, ITGAX, PTPRN2, and CLCN4; BCOR, UBTD1, RPS4Y2, DIP2C, MGC16121, and PTPRN2; BCOR, UBTD1, RPS4Y2, DIP2C, MGC16121, and CLCN4; BCOR, UBTD1, RPS4Y2, DIP2C, PTPRN2, and CLCN4; BCOR, UBTD1, RPS4Y2, MGC16121, PTPRN2, and CLCN4; BCOR, UBTD1, PPAPDC1A, ITGAX, DIP2C, and MGC16121; BCOR, UBTD1, PPAPDC1A, ITGAX, DIP2C, and PTPRN2; BCOR, UBTD1, PPAPDC1A, ITGAX, DIP2C, and CLCN4; BCOR, UBTD1, PPAPDC1A, ITGAX, MGC16121, and PTPRN2; BCOR, UBTD1, PPAPDC1A, ITGAX, MGC16121, and CLCN4; BCOR, UBTD1, PPAPDC1A, ITGAX, PTPRN2, and CLCN4; BCOR, UBTD1, PPAPDC1A, DIP2C, MGC16121, and PTPRN2; BCOR, UBTD1, PPAPDC1A, DIP2C, MGC16121, and CLCN4; BCOR, UBTD1, PPAPDC1A, DIP2C, PTPRN2, and CLCN4; BCOR, UBTD1, PPAPDC1A, MGC16121, PTPRN2, and CLCN4; BCOR, UBTD1, ITGAX, DIP2C, MGC16121, and PTPRN2; BCOR, UBTD1, ITGAX, DIP2C, MGC16121, and CLCN4; BCOR, UBTD1, ITGAX, DIP2C, PTPRN2, and CLCN4; BCOR, UBTD1, ITGAX, MGC16121, PTPRN2, and CLCN4; BCOR, UBTD1, DIP2C, MGC16121, PTPRN2, and CLCN4; BCOR, LOC285375, RPS4Y2, PPAPDC1A, ITGAX, and DIP2C; BCOR, LOC285375, RPS4Y2, PPAPDC1A, ITGAX, and MGC16121; BCOR, LOC285375, RPS4Y2, PPAPDC1A, ITGAX, and PTPRN2; BCOR, LOC285375, RPS4Y2, PPAPDC1A, ITGAX, and CLCN4; BCOR, LOC285375, RPS4Y2, PPAPDC1A, DIP2C, and MGC16121; BCOR, LOC285375, RPS4Y2, PPAPDC1A, DIP2C, and PTPRN2; BCOR, LOC285375, RPS4Y2, PPAPDC1A, DIP2C, and CLCN4; BCOR, LOC285375, RPS4Y2, PPAPDC1A, MGC16121, and PTPRN2; BCOR, LOC285375, RPS4Y2, PPAPDC1A, MGC16121, and CLCN4; BCOR, LOC285375, RPS4Y2, PPAPDC1A, PTPRN2, and CLCN4; BCOR, LOC285375, RPS4Y2, ITGAX, DIP2C, and MGC16121; BCOR, LOC285375, RPS4Y2, ITGAX, DIP2C, and PTPRN2; BCOR, LOC285375, RPS4Y2, ITGAX, DIP2C, and CLCN4; BCOR, LOC285375, RPS4Y2, ITGAX, MGC16121, and PTPRN2; BCOR, LOC285375, RPS4Y2, ITGAX, MGC16121, and CLCN4; BCOR, LOC285375, RPS4Y2, ITGAX, PTPRN2, and CLCN4; BCOR, LOC285375, RPS4Y2, DIP2C, MGC16121, and PTPRN2; BCOR, LOC285375, RPS4Y2, DIP2C, MGC16121, and CLCN4; BCOR, LOC285375, RPS4Y2, DIP2C, PTPRN2, and CLCN4; BCOR, LOC285375, RPS4Y2, MGC16121, PTPRN2, and CLCN4; BCOR, LOC285375, PPAPDC1A, ITGAX, DIP2C, and MGC16121; BCOR, LOC285375, PPAPDC1A, ITGAX, DIP2C, and PTPRN2; BCOR, LOC285375, PPAPDC1A, ITGAX, DIP2C, and CLCN4; BCOR, LOC285375, PPAPDC1A, ITGAX, MGC16121, and PTPRN2; BCOR, LOC285375, PPAPDC1A, ITGAX, MGC16121, and CLCN4; BCOR, LOC285375, PPAPDC1A, ITGAX, PTPRN2, and CLCN4; BCOR, LOC285375, PPAPDC1A, DIP2C, MGC16121, and PTPRN2; BCOR, LOC285375, PPAPDC1A, DIP2C, MGC16121, and CLCN4; BCOR, LOC285375, PPAPDC1A, DIP2C, PTPRN2, and CLCN4; BCOR, LOC285375, PPAPDC1A, MGC16121, PTPRN2, and CLCN4; BCOR, LOC285375, ITGAX, DIP2C, MGC16121, and PTPRN2; BCOR, LOC285375, ITGAX, DIP2C, MGC16121, and CLCN4; BCOR, LOC285375, ITGAX, DIP2C, PTPRN2, and CLCN4; BCOR, LOC285375, ITGAX, MGC16121, PTPRN2, and CLCN4; BCOR, LOC285375, DIP2C, MGC16121, PTPRN2, and CLCN4; BCOR, RPS4Y2, PPAPDC1A, ITGAX, DIP2C, and MGC16121; BCOR, RPS4Y2, PPAPDC1A, ITGAX, DIP2C, and PTPRN2; BCOR, RPS4Y2, PPAPDC1A, ITGAX, DIP2C, and CLCN4; BCOR, RPS4Y2, PPAPDC1A, ITGAX, MGC16121, and PTPRN2; BCOR, RPS4Y2, PPAPDC1A, ITGAX, MGC16121, and CLCN4; BCOR, RPS4Y2, PPAPDC1A, ITGAX, PTPRN2, and CLCN4; BCOR, RPS4Y2, PPAPDC1A, DIP2C, MGC16121, and PTPRN2; BCOR, RPS4Y2, PPAPDC1A, DIP2C, MGC16121, and CLCN4; BCOR, RPS4Y2, PPAPDC1A, DIP2C, PTPRN2, and CLCN4; BCOR, RPS4Y2, PPAPDC1A, MGC16121, PTPRN2, and CLCN4; BCOR, RPS4Y2, ITGAX, DIP2C, MGC16121, and PTPRN2; BCOR, RPS4Y2, ITGAX, DIP2C, MGC16121, and CLCN4; BCOR, RPS4Y2, ITGAX, DIP2C, PTPRN2, and CLCN4; BCOR, RPS4Y2, ITGAX, MGC16121, PTPRN2, and CLCN4; BCOR, RPS4Y2, DIP2C, MGC16121, PTPRN2, and CLCN4; BCOR, PPAPDC1A, ITGAX, DIP2C, MGC16121, and PTPRN2; BCOR, PPAPDC1A, ITGAX, DIP2C, MGC16121, and CLCN4; BCOR, PPAPDC1A, ITGAX, DIP2C, PTPRN2, and CLCN4; BCOR, PPAPDC1A, ITGAX, MGC16121, PTPRN2, and CLCN4; BCOR, PPAPDC1A, DIP2C, MGC16121, PTPRN2, and CLCN4; BCOR, ITGAX, DIP2C, MGC16121, PTPRN2, and CLCN4; UBTD1, LOC285375, RPS4Y2, PPAPDC1A, ITGAX, and DIP2C; UBTD1, LOC285375, RPS4Y2, PPAPDC1A, ITGAX, and MGC16121; UBTD1, LOC285375, RPS4Y2, PPAPDC1A, ITGAX, and PTPRN2; UBTD1, LOC285375, RPS4Y2, PPAPDC1A, ITGAX, and CLCN4; UBTD1, LOC285375, RPS4Y2, PPAPDC1A, DIP2C, and MGC16121; UBTD1, LOC285375, RPS4Y2, PPAPDC1A, DIP2C, and PTPRN2; UBTD1, LOC285375, RPS4Y2, PPAPDC1A, DIP2C, and CLCN4; UBTD1, LOC285375, RPS4Y2, PPAPDC1A, MGC16121, and PTPRN2; UBTD1, LOC285375, RPS4Y2, PPAPDC1A, MGC16121, and CLCN4; UBTD1, LOC285375, RPS4Y2, PPAPDC1A, PTPRN2, and CLCN4; UBTD1, LOC285375, RPS4Y2, ITGAX, DIP2C, and MGC16121; UBTD1, LOC285375, RPS4Y2, ITGAX, DIP2C, and PTPRN2; UBTD1, LOC285375, RPS4Y2, ITGAX, DIP2C, and CLCN4; UBTD1, LOC285375, RPS4Y2, ITGAX, MGC16121, and PTPRN2; UBTD1, LOC285375, RPS4Y2, ITGAX, MGC16121, and CLCN4; UBTD1, LOC285375, RPS4Y2, ITGAX, PTPRN2, and CLCN4; UBTD1, LOC285375, RPS4Y2, DIP2C, MGC16121, and PTPRN2; UBTD1, LOC285375, RPS4Y2, DIP2C, MGC16121, and CLCN4; UBTD1, LOC285375, RPS4Y2, DIP2C, PTPRN2, and CLCN4; UBTD1, LOC285375, RPS4Y2, MGC16121, PTPRN2, and CLCN4; UBTD1, LOC285375, PPAPDC1A, ITGAX, DIP2C, and MGC16121; UBTD1, LOC285375, PPAPDC1A, ITGAX, DIP2C, and PTPRN2; UBTD1, LOC285375, PPAPDC1A, ITGAX, DIP2C, and CLCN4; UBTD1, LOC285375, PPAPDC1A, ITGAX, MGC16121, and PTPRN2; UBTD1, LOC285375, PPAPDC1A, ITGAX, MGC16121, and CLCN4; UBTD1, LOC285375, PPAPDC1A, ITGAX, PTPRN2, and CLCN4; UBTD1, LOC285375, PPAPDC1A, DIP2C, MGC16121, and PTPRN2; UBTD1, LOC285375, PPAPDC1A, DIP2C, MGC16121, and CLCN4; UBTD1, LOC285375, PPAPDC1A, DIP2C, PTPRN2, and CLCN4; UBTD1, LOC285375, PPAPDC1A, MGC16121, PTPRN2, and CLCN4; UBTD1, LOC285375, ITGAX, DIP2C, MGC16121, and PTPRN2; UBTD1, LOC285375, ITGAX, DIP2C, MGC16121, and CLCN4; UBTD1, LOC285375, ITGAX, DIP2C, PTPRN2, and CLCN4; UBTD1, LOC285375, ITGAX, MGC16121, PTPRN2, and CLCN4; UBTD1, LOC285375, DIP2C, MGC16121, PTPRN2, and CLCN4; UBTD1, RPS4Y2, PPAPDC1A, ITGAX, DIP2C, and MGC16121; UBTD1, RPS4Y2, PPAPDC1A, ITGAX, DIP2C, and PTPRN2; UBTD1, RPS4Y2, PPAPDC1A, ITGAX, DIP2C, and CLCN4; UBTD1, RPS4Y2, PPAPDC1A, ITGAX, MGC16121, and PTPRN2; UBTD1, RPS4Y2, PPAPDC1A, ITGAX, MGC16121, and CLCN4; UBTD1, RPS4Y2, PPAPDC1A, ITGAX, PTPRN2, and CLCN4; UBTD1, RPS4Y2, PPAPDC1A, DIP2C, MGC16121, and PTPRN2; UBTD1, RPS4Y2, PPAPDC1A, DIP2C, MGC16121, and CLCN4; UBTD1, RPS4Y2, PPAPDC1A, DIP2C, PTPRN2, and CLCN4; UBTD1, RPS4Y2, PPAPDC1A, MGC16121, PTPRN2, and CLCN4; UBTD1, RPS4Y2, ITGAX, DIP2C, MGC16121, and PTPRN2; UBTD1, RPS4Y2, ITGAX, DIP2C, MGC16121, and CLCN4; UBTD1, RPS4Y2, ITGAX, DIP2C, PTPRN2, and CLCN4; UBTD1, RPS4Y2, ITGAX, MGC16121, PTPRN2, and CLCN4; UBTD1, RPS4Y2, DIP2C, MGC16121, PTPRN2, and CLCN4; UBTD1, PPAPDC1A, ITGAX, DIP2C, MGC16121, and PTPRN2; UBTD1, PPAPDC1A, ITGAX, DIP2C, MGC16121, and CLCN4; UBTD1, PPAPDC1A, ITGAX, DIP2C, PTPRN2, and CLCN4; UBTD1, PPAPDC1A, ITGAX, MGC16121, PTPRN2, and CLCN4; UBTD1, PPAPDC1A, DIP2C, MGC16121, PTPRN2, C and LCN4; UBTD1, ITGAX, DIP2C, MGC16121, PTPRN2, and CLCN4; LOC285375, RPS4Y2, PPAPDC1A, ITGAX, DIP2C, and MGC16121; LOC285375, RPS4Y2, PPAPDC1A, ITGAX, DIP2C, and PTPRN2; LOC285375, RPS4Y2, PPAPDC1A, ITGAX, DIP2C, and CLCN4; LOC285375, RPS4Y2, PPAPDC1A, ITGAX, MGC16121, and PTPRN2; LOC285375, RPS4Y2, PPAPDC1A, ITGAX, MGC16121, and CLCN4; LOC285375, RPS4Y2, PPAPDC1A, ITGAX, PTPRN2, and CLCN4; LOC285375, RPS4Y2, PPAPDC1A, DIP2C, MGC16121, and PTPRN2; LOC285375, RPS4Y2, PPAPDC1A, DIP2C, MGC16121, and CLCN4; LOC285375, RPS4Y2, PPAPDC1A, DIP2C, PTPRN2, and CLCN4; LOC285375, RPS4Y2, PPAPDC1A, MGC16121, PTPRN2, and CLCN4; LOC285375, RPS4Y2, ITGAX, DIP2C, MGC16121, and PTPRN2; LOC285375, RPS4Y2, ITGAX, DIP2C, MGC16121, and CLCN4; LOC285375, RPS4Y2, ITGAX, DIP2C, PTPRN2, and CLCN4; LOC285375, RPS4Y2, ITGAX, MGC16121, PTPRN2, and CLCN4; LOC285375, RPS4Y2, DIP2C, MGC16121, PTPRN2, and CLCN4; LOC285375, PPAPDC1A, ITGAX, DIP2C, MGC16121, and PTPRN2; LOC285375, PPAPDC1A, ITGAX, DIP2C, MGC16121, C and LCN4; LOC285375, PPAPDC1A, ITGAX, DIP2C, PTPRN2, and CLCN4; LOC285375, PPAPDC1A, ITGAX, MGC16121, PTPRN2, and CLCN4; LOC285375, PPAPDC1A, DIP2C, MGC16121, PTPRN2, and CLCN4; LOC285375, ITGAX, DIP2C, MGC16121, PTPRN2, and CLCN4; RPS4Y2, PPAPDC1A, ITGAX, DIP2C, MGC16121, and PTPRN2; RPS4Y2, PPAPDC1A, ITGAX, DIP2C, MGC16121, and CLCN4; RPS4Y2, PPAPDC1A, ITGAX, DIP2C, PTPRN2, and CLCN4; RPS4Y2, PPAPDC1A, ITGAX, MGC16121, PTPRN2, and CLCN4; RPS4Y2, PPAPDC1A, DIP2C, MGC16121, PTPRN2, and CLCN4; RPS4Y2, ITGAX, DIP2C, MGC16121, PTPRN2, and CLCN4; and PPAPDC1A, ITGAX, DIP2C, MGC16121, PTPRN2, and CLCN4. In particular embodiments, the markers include BCOR, UBTD1, LOC285375, RPS4Y2, PPAPDC1A; BCOR, UBTD1, LOC285375, RPS4Y2, ITGAX; BCOR, UBTD1, LOC285375, RPS4Y2, DIP2C; BCOR, UBTD1, LOC285375, RPS4Y2, MGC16121; BCOR, UBTD1, LOC285375, RPS4Y2, PTPRN2; BCOR, UBTD1, LOC285375, RPS4Y2, CLCN4; BCOR, UBTD1, LOC285375, PPAPDC1A, ITGAX; BCOR, UBTD1, LOC285375, PPAPDC1A, DIP2C; BCOR, UBTD1, LOC285375, PPAPDC1A, MGC16121; BCOR, UBTD1, LOC285375, PPAPDC1A, PTPRN2; BCOR, UBTD1, LOC285375, PPAPDC1A, CLCN4; BCOR, UBTD1, LOC285375, ITGAX, DIP2C; BCOR, UBTD1, LOC285375, ITGAX, MGC16121; BCOR, UBTD1, LOC285375, ITGAX, PTPRN2; BCOR, UBTD1, LOC285375, ITGAX, CLCN4; BCOR, UBTD1, LOC285375, DIP2C, MGC16121; BCOR, UBTD1, LOC285375, DIP2C, PTPRN2; BCOR, UBTD1, LOC285375, DIP2C, CLCN4; BCOR, UBTD1, LOC285375, MGC16121, PTPRN2; BCOR, UBTD1, LOC285375, MGC16121, CLCN4; BCOR, UBTD1, LOC285375, PTPRN2, CLCN4; BCOR, UBTD1, RPS4Y2, PPAPDC1A, ITGAX; BCOR, UBTD1, RPS4Y2, PPAPDC1A, DIP2C; BCOR, UBTD1, RPS4Y2, PPAPDC1A, MGC16121; BCOR, UBTD1, RPS4Y2, PPAPDC1A, PTPRN2; BCOR, UBTD1, RPS4Y2, PPAPDC1A, CLCN4; BCOR, UBTD1, RPS4Y2, ITGAX, DIP2C; BCOR, UBTD1, RPS4Y2, ITGAX, MGC16121; BCOR, UBTD1, RPS4Y2, ITGAX, PTPRN2; BCOR, UBTD1, RPS4Y2, ITGAX, CLCN4; BCOR, UBTD1, RPS4Y2, DIP2C, MGC16121; BCOR, UBTD1, RPS4Y2, DIP2C, PTPRN2; BCOR, UBTD1, RPS4Y2, DIP2C, CLCN4; BCOR, UBTD1, RPS4Y2, MGC16121, PTPRN2; BCOR, UBTD1, RPS4Y2, MGC16121, CLCN4; BCOR, UBTD1, RPS4Y2, PTPRN2, CLCN4; BCOR, UBTD1, PPAPDC1A, ITGAX, DIP2C; BCOR, UBTD1, PPAPDC1A, ITGAX, MGC16121; BCOR, UBTD1, PPAPDC1A, ITGAX, PTPRN2; BCOR, UBTD1, PPAPDC1A, ITGAX, CLCN4; BCOR, UBTD1, PPAPDC1A, DIP2C, MGC16121; BCOR, UBTD1, PPAPDC1A, DIP2C, PTPRN2; BCOR, UBTD1, PPAPDC1A, DIP2C, CLCN4; BCOR, UBTD1, PPAPDC1A, MGC16121, PTPRN2; BCOR, UBTD1, PPAPDC1A, MGC16121, CLCN4; BCOR, UBTD1, PPAPDC1A, PTPRN2, CLCN4; BCOR, UBTD1, ITGAX, DIP2C, MGC16121; BCOR, UBTD1, ITGAX, DIP2C, PTPRN2; BCOR, UBTD1, ITGAX, DIP2C, CLCN4; BCOR, UBTD1, DIP2C, MGC16121, PTPRN2; BCOR, UBTD1, DIP2C, MGC16121, CLCN4; BCOR, UBTD1, DIP2C, PTPRN2, CLCN4; BCOR, UBTD1, MGC16121, PTPRN2, CLCN4; BCOR, LOC285375, RPS4Y2, PPAPDC1A, ITGAX; BCOR, LOC285375, RPS4Y2, PPAPDC1A, DIP2C; BCOR, LOC285375, RPS4Y2, PPAPDC1A, MGC16121; BCOR, LOC285375, RPS4Y2, PPAPDC1A, MGC16121; BCOR, LOC285375, RPS4Y2, PPAPDC1A, PTPRN2; BCOR, LOC285375, RPS4Y2, PPAPDC1A, PTPRN2; BCOR, LOC285375, RPS4Y2, PPAPDC1A, CLCN4; BCOR, LOC285375, RPS4Y2, PPAPDC1A, CLCN4; BCOR, LOC285375, RPS4Y2, ITGAX, DIP2C; BCOR, LOC285375, RPS4Y2, ITGAX, DIP2C; BCOR, LOC285375, RPS4Y2, ITGAX, MGC16121; BCOR, LOC285375, RPS4Y2, ITGAX, MGC16121; BCOR, LOC285375, RPS4Y2, ITGAX, PTPRN2; BCOR, LOC285375, RPS4Y2, ITGAX, PTPRN2; BCOR, LOC285375, RPS4Y2, ITGAX, CLCN4; BCOR, LOC285375, RPS4Y2, ITGAX, CLCN4; BCOR, LOC285375, RPS4Y2, DIP2C, MGC16121; BCOR, LOC285375, RPS4Y2, DIP2C, MGC16121; BCOR, LOC285375, RPS4Y2, DIP2C, PTPRN2; BCOR, LOC285375, RPS4Y2, DIP2C, PTPRN2; BCOR, LOC285375, RPS4Y2, DIP2C, CLCN4; BCOR, LOC285375, RPS4Y2, DIP2C, CLCN4; BCOR, LOC285375, RPS4Y2, MGC16121, PTPRN2; BCOR, LOC285375, RPS4Y2, MGC16121, PTPRN2; BCOR, LOC285375, RPS4Y2, MGC16121, CLCN4; BCOR, LOC285375, RPS4Y2, MGC16121, CLCN4; BCOR, LOC285375, RPS4Y2, PTPRN2, CLCN4; BCOR, LOC285375, RPS4Y2, PTPRN2, CLCN4; BCOR, LOC285375, PPAPDC1A, ITGAX, DIP2C; BCOR, LOC285375, PPAPDC1A, ITGAX, DIP2C; BCOR, LOC285375, PPAPDC1A, ITGAX, MGC16121; BCOR, LOC285375, PPAPDC1A, ITGAX, PTPRN2; BCOR, LOC285375, PPAPDC1A, ITGAX, CLCN4; BCOR, LOC285375, PPAPDC1A, DIP2C, MGC16121; BCOR, LOC285375, PPAPDC1A, DIP2C, MGC16121; BCOR, LOC285375, PPAPDC1A, DIP2C, PTPRN2; BCOR, LOC285375, PPAPDC1A, DIP2C, PTPRN2; BCOR, LOC285375, PPAPDC1A, DIP2C, CLCN4; BCOR, LOC285375, PPAPDC1A, DIP2C, CLCN4; BCOR, LOC285375, PPAPDC1A, MGC16121, PTPRN2; BCOR, LOC285375, PPAPDC1A, MGC16121, PTPRN2; BCOR, LOC285375, PPAPDC1A, MGC16121, CLCN4; BCOR, LOC285375, PPAPDC1A, MGC16121, CLCN4; BCOR, LOC285375, PPAPDC1A, PTPRN2, CLCN4; BCOR, LOC285375, ITGAX, DIP2C, MGC16121; BCOR, LOC285375, ITGAX, DIP2C, MGC16121; BCOR, LOC285375, ITGAX, DIP2C, PTPRN2; BCOR, LOC285375, ITGAX, DIP2C, PTPRN2; BCOR, LOC285375, ITGAX, DIP2C, CLCN4; BCOR, LOC285375, ITGAX, DIP2C, CLCN4; BCOR, LOC285375, ITGAX, MGC16121, PTPRN2; BCOR, LOC285375, ITGAX, MGC16121, PTPRN2; BCOR, LOC285375, ITGAX, MGC16121, CLCN4; BCOR, LOC285375, ITGAX, MGC16121, CLCN4; BCOR, LOC285375, ITGAX, PTPRN2, CLCN4; BCOR, LOC285375, ITGAX, PTPRN2, CLCN4; BCOR, LOC285375, DIP2C, MGC16121, PTPRN2; BCOR, LOC285375, DIP2C, MGC16121, PTPRN2; BCOR, LOC285375, DIP2C, MGC16121, CLCN4; BCOR, LOC285375, DIP2C, MGC16121, CLCN4; BCOR, LOC285375, DIP2C, PTPRN2, CLCN4; BCOR, LOC285375, DIP2C, PTPRN2, CLCN4; BCOR, LOC285375, MGC16121, PTPRN2, CLCN4; BCOR, LOC285375, MGC16121, PTPRN2, CLCN4; BCOR, RPS4Y2, PPAPDC1A, ITGAX, DIP2C; BCOR, RPS4Y2, PPAPDC1A, ITGAX, DIP2C; BCOR, RPS4Y2, PPAPDC1A, ITGAX, MGC16121; BCOR, RPS4Y2, PPAPDC1A, ITGAX, MGC16121; BCOR, RPS4Y2, PPAPDC1A, ITGAX, PTPRN2; BCOR, RPS4Y2, PPAPDC1A, ITGAX, PTPRN2; BCOR, RPS4Y2, PPAPDC1A, ITGAX, CLCN4; BCOR, RPS4Y2, PPAPDC1A, ITGAX, CLCN4; BCOR, RPS4Y2, PPAPDC1A, DIP2C, MGC16121; BCOR, RPS4Y2, PPAPDC1A, DIP2C, MGC16121; BCOR, RPS4Y2, PPAPDC1A, DIP2C, PTPRN2; BCOR, RPS4Y2, PPAPDC1A, DIP2C, PTPRN2; BCOR, RPS4Y2, PPAPDC1A, DIP2C, CLCN4; BCOR, RPS4Y2, PPAPDC1A, DIP2C, CLCN4; BCOR, RPS4Y2, PPAPDC1A, MGC16121, PTPRN2; BCOR, RPS4Y2, PPAPDC1A, MGC16121, PTPRN2; BCOR, RPS4Y2, PPAPDC1A, MGC16121, CLCN4; BCOR, RPS4Y2, PPAPDC1A, MGC16121, CLCN4; BCOR, RPS4Y2, PPAPDC1A, PTPRN2, CLCN4; BCOR, RPS4Y2, PPAPDC1A, PTPRN2, CLCN4; BCOR, RPS4Y2, ITGAX, DIP2C, MGC16121; BCOR, RPS4Y2, ITGAX, DIP2C, MGC16121; BCOR, RPS4Y2, ITGAX, DIP2C, PTPRN2; BCOR, RPS4Y2, ITGAX, DIP2C, PTPRN2; BCOR, RPS4Y2, ITGAX, DIP2C, CLCN4; BCOR, RPS4Y2, ITGAX, DIP2C, CLCN4; BCOR, RPS4Y2, ITGAX, MGC16121, PTPRN2; BCOR, RPS4Y2, ITGAX, MGC16121, PTPRN2; BCOR, RPS4Y2, ITGAX, MGC16121, CLCN4; BCOR, RPS4Y2, ITGAX, MGC16121, CLCN4; BCOR, RPS4Y2, ITGAX, PTPRN2, CLCN4; BCOR, RPS4Y2, ITGAX, PTPRN2, CLCN4; BCOR, RPS4Y2, DIP2C, MGC16121, PTPRN2; BCOR, RPS4Y2, DIP2C, MGC16121, PTPRN2; BCOR, RPS4Y2, DIP2C, MGC16121, CLCN4; BCOR, RPS4Y2, DIP2C, MGC16121, CLCN4; BCOR, RPS4Y2, DIP2C, PTPRN2, CLCN4; BCOR, RPS4Y2, DIP2C, PTPRN2, CLCN4; BCOR, RPS4Y2, MGC16121, PTPRN2, CLCN4; BCOR, RPS4Y2, MGC16121, PTPRN2, CLCN4; BCOR, PPAPDC1A, ITGAX, DIP2C, MGC16121; BCOR, PPAPDC1A, ITGAX, DIP2C, MGC16121; BCOR, PPAPDC1A, ITGAX, DIP2C, PTPRN2; BCOR, PPAPDC1A, ITGAX, DIP2C, PTPRN2; BCOR, PPAPDC1A, ITGAX, DIP2C, CLCN4; BCOR, PPAPDC1A, ITGAX, DIP2C, CLCN4; BCOR, PPAPDC1A, ITGAX, MGC16121, PTPRN2; BCOR, PPAPDC1A, ITGAX, MGC16121, PTPRN2; BCOR, PPAPDC1A, ITGAX, MGC16121, CLCN4; BCOR, PPAPDC1A, ITGAX, MGC16121, CLCN4; BCOR, PPAPDC1A, ITGAX, PTPRN2, CLCN4; BCOR, PPAPDC1A, ITGAX, PTPRN2, CLCN4; BCOR, PPAPDC1A, DIP2C, MGC16121, PTPRN2; BCOR, PPAPDC1A, DIP2C, MGC16121, CLCN4; BCOR, PPAPDC1A, DIP2C, PTPRN2, CLCN4; BCOR, PPAPDC1A, MGC16121, PTPRN2, CLCN4; BCOR, ITGAX, DIP2C, MGC16121, PTPRN2; BCOR, ITGAX, DIP2C, MGC16121, PTPRN2; BCOR, ITGAX, DIP2C, MGC16121, CLCN4; BCOR, ITGAX, DIP2C, MGC16121, CLCN4; BCOR, ITGAX, DIP2C, PTPRN2, CLCN4; BCOR, ITGAX, DIP2C, PTPRN2, CLCN4; BCOR, ITGAX, MGC16121, PTPRN2, CLCN4; BCOR, DIP2C, MGC16121, PTPRN2, CLCN4; BCOR, DIP2C, MGC16121, PTPRN2, CLCN4; UBTD1, LOC285375, RPS4Y2, PPAPDC1A, ITGAX; UBTD1, LOC285375, RPS4Y2, PPAPDC1A, ITGAX; UBTD1, LOC285375, RPS4Y2, PPAPDC1A, DIP2C; UBTD1, LOC285375, RPS4Y2, PPAPDC1A, MGC16121; UBTD1, LOC285375, RPS4Y2, PPAPDC1A, PTPRN2; UBTD1, LOC285375, RPS4Y2, PPAPDC1A, CLCN4; UBTD1, LOC285375, RPS4Y2, ITGAX, DIP2C; UBTD1, LOC285375, RPS4Y2, ITGAX, MGC16121; UBTD1, LOC285375, RPS4Y2, ITGAX, PTPRN2; UBTD1, LOC285375, RPS4Y2, ITGAX, CLCN4; UBTD1, LOC285375, RPS4Y2, DIP2C, MGC16121; UBTD1, LOC285375, RPS4Y2, DIP2C, PTPRN2; UBTD1, LOC285375, RPS4Y2, DIP2C, CLCN4; UBTD1, LOC285375, RPS4Y2, MGC16121, PTPRN2; UBTD1, LOC285375, RPS4Y2, MGC16121, CLCN4; UBTD1, LOC285375, RPS4Y2, PTPRN2, CLCN4; UBTD1, LOC285375, PPAPDC1A, ITGAX, DIP2C; UBTD1, LOC285375, PPAPDC1A, ITGAX, MGC16121; UBTD1, LOC285375, PPAPDC1A, ITGAX, PTPRN2; UBTD1, LOC285375, PPAPDC1A, ITGAX, CLCN4; UBTD1, LOC285375, PPAPDC1A, DIP2C, MGC16121; UBTD1, LOC285375, PPAPDC1A, DIP2C, PTPRN2; UBTD1, LOC285375, PPAPDC1A, DIP2C, CLCN4; UBTD1, LOC285375, PPAPDC1A, MGC16121, PTPRN2; UBTD1, LOC285375, PPAPDC1A, MGC16121, CLCN4; UBTD1, LOC285375, PPAPDC1A, PTPRN2, CLCN4; UBTD1, LOC285375, ITGAX, DIP2C, MGC16121; UBTD1, LOC285375, ITGAX, DIP2C, PTPRN2; UBTD1, LOC285375, ITGAX, DIP2C, CLCN4; UBTD1, LOC285375, ITGAX, MGC16121, PTPRN2; UBTD1, LOC285375, ITGAX, MGC16121, CLCN4; UBTD1, LOC285375, ITGAX, PTPRN2, CLCN4; UBTD1, LOC285375, DIP2C, MGC16121, PTPRN2; UBTD1, LOC285375, DIP2C, MGC16121, CLCN4; UBTD1, LOC285375, DIP2C, PTPRN2, CLCN4; UBTD1, LOC285375, MGC16121, PTPRN2, CLCN4; UBTD1, RPS4Y2, PPAPDC1A, ITGAX, DIP2C; UBTD1, RPS4Y2, PPAPDC1A, ITGAX, MGC16121; UBTD1, RPS4Y2, PPAPDC1A, ITGAX, PTPRN2; UBTD1, RPS4Y2, PPAPDC1A, ITGAX, CLCN4; UBTD1, RPS4Y2, PPAPDC1A, DIP2C, MGC16121; UBTD1, RPS4Y2, PPAPDC1A, DIP2C, PTPRN2; UBTD1, RPS4Y2, PPAPDC1A, DIP2C, CLCN4; UBTD1, RPS4Y2, PPAPDC1A, MGC16121, PTPRN2; UBTD1, RPS4Y2, PPAPDC1A, MGC16121, CLCN4; UBTD1, RPS4Y2, PPAPDC1A, PTPRN2, CLCN4; UBTD1, RPS4Y2, ITGAX, DIP2C, MGC16121; UBTD1, RPS4Y2, ITGAX, DIP2C, PTPRN2; UBTD1, RPS4Y2, ITGAX, DIP2C, CLCN4; UBTD1, RPS4Y2, ITGAX, MGC16121, PTPRN2; UBTD1, RPS4Y2, ITGAX, MGC16121, CLCN4; UBTD1, RPS4Y2, ITGAX, PTPRN2, CLCN4; UBTD1, RPS4Y2, DIP2C, MGC16121, PTPRN2; UBTD1, RPS4Y2, DIP2C, MGC16121, CLCN4; UBTD1, RPS4Y2, DIP2C, PTPRN2, CLCN4; UBTD1, RPS4Y2, MGC16121, PTPRN2, CLCN4; UBTD1, PPAPDC1A, ITGAX, DIP2C, MGC16121; UBTD1, PPAPDC1A, ITGAX, DIP2C, PTPRN2; UBTD1, PPAPDC1A, ITGAX, DIP2C, CLCN4; UBTD1, PPAPDC1A, ITGAX, MGC16121, PTPRN2; UBTD1, PPAPDC1A, ITGAX, MGC16121, CLCN4; UBTD1, PPAPDC1A, ITGAX, PTPRN2, CLCN4; UBTD1, PPAPDC1A, DIP2C, MGC16121, PTPRN2; UBTD1, PPAPDC1A, DIP2C, MGC16121, CLCN4; UBTD1, PPAPDC1A, DIP2C, PTPRN2, CLCN4; UBTD1, PPAPDC1A, MGC16121, PTPRN2, CLCN4; UBTD1, ITGAX, DIP2C, MGC16121, PTPRN2; UBTD1, ITGAX, DIP2C, MGC16121, CLCN4; UBTD1, ITGAX, DIP2C, PTPRN2, CLCN4; or UBTD1, DIP2C, MGC16121, PTPRN2, CLCN4.

In particular embodiments, the markers include RAP1GAP2, UBTD1, MAMLD1, and C8orf75. In particular embodiments, the markers include UBTD1, MAMLD1, and C8orf75. In particular embodiments, the markers include RAP1GAP2, MAMLD1, and C8orf75. In particular embodiments, the markers include RAP1GAP2, UBTD1, and C8orf75. In particular embodiments, the markers include RAP1GAP2, UBTD1, and MAMLD1.

In other embodiments, the markers include BCOR in combination with two, three, or four markers selected from: PTPRN2, TUBA3D, PDE9A, and LOC284412; PTPRN2 in combination with two, three, or four markers selected from: BCOR, TUBA3D, PDE9A, and LOC284412; TUBA3D in combination with two, three, or four markers selected from: BCOR, PTPRN2, PDE9A, and LOC284412; PDE9A in combination with two, three, or four markers selected from: BCOR, PTPRN2, TUBA3D, and LOC284412; or LOC284412 in combination with two, three, or four markers selected from: BCOR, PTPRN2, TUBA3D, and PDE9A.

In other embodiments, the markers include GPM6B in combination with two or three markers selected from: NDUFA10, PDE9A, and LOC284412; NDUFA10 in combination with two or three markers selected from: GPM6B, PDE9A, and LOC284412; PDE9A in combination with two or three markers selected from: GPM6B, NDUFA10, and LOC284412; and LOC284412in combination with two or three markers selected from: GPM6B, NDUFA10, and PDE9A.

In other embodiments, the markers include BCOR in combination with two, three, four, five, six, seven, eight or nine markers selected from: UBTD1, LOC285375, RPS4Y2, PPAPDC1A, ITGAX, DIP2C, MGC16121, PTPRN2, and CLCN4; UBTD1 in combination with two, three, four, five, six, seven, eight or nine markers selected from: BCOR, LOC285375, RPS4Y2, PPAPDC1A, ITGAX, DIP2C, MGC16121, PTPRN2, and CLCN4; LOC285375 in combination with two, three, four, five, six, seven, eight or nine markers selected from: BCOR, UBTD1, RPS4Y2, PPAPDC1A, ITGAX, DIP2C, MGC16121, PTPRN2, and CLCN4; RPS4Y2 in combination with two, three, four, five, six, seven, eight or nine markers selected from: BCOR, UBTD1, LOC285375, PPAPDC1A, ITGAX, DIP2C, MGC16121, PTPRN2, and CLCN4; PPAPDC1A in combination with two, three, four, five, six, seven, eight or nine markers selected from: BCOR, UBTD1, LOC285375, RPS4Y2, ITGAX, DIP2C, MGC16121, PTPRN2, and CLCN4; ITGAX in combination with two, three, four, five, six, seven, eight or nine markers selected from: BCOR, UBTD1, LOC285375, RPS4Y2, PPAPDC1A, DIP2C, MGC16121, PTPRN2, and CLCN4; DIP2C in combination with two, three, four, five, six, seven, eight or nine markers selected from: BCOR, UBTD1, LOC285375, RPS4Y2, PPAPDC1A, ITGAX, MGC16121, PTPRN2, and CLCN4; MGC16121 in combination with two, three, four, five, six, seven, eight or nine markers selected from: BCOR, UBTD1, LOC285375, RPS4Y2, PPAPDC1A, ITGAX, DIP2C, PTPRN2, and CLCN4; PTPRN2 in combination with two, three, four, five, six, seven, eight or nine markers selected from: BCOR, UBTD1, LOC285375, RPS4Y2, PPAPDC1A, ITGAX, DIP2C, MGC16121, and CLCN4; or CLCN4 in combination with two, three, four, five, six, seven, eight or nine markers selected from: BCOR, UBTD1, LOC285375, RPS4Y2, PPAPDC1A, ITGAX, DIP2C, MGC16121, and PTPRN2.

In other embodiments, the markers include RAP1GAP2 in combination with two, three, four, five, six, seven, eight or nine markers selected from:, UBTD1, MAMLD1, and C8orf75; UBTD1 in combination with two, three, four, five, six, seven, eight or nine markers selected from: RAP1GAP2, MAMLD1, and C8orf75; MAMLD1 in combination with two, three, four, five, six, seven, eight or nine markers selected from: RAP1GAP2, UBTD1, and C8orf75; or C8orf75 in combination with two, three, four, five, six, seven, eight or nine markers selected from: RAP1GAP2, UBTD1, and MAMLD1.

In other embodiments, the markers exclude BCOR. In other embodiments, the markers exclude C8orf75. In other embodiments, the markers exclude CLCN4. In other embodiments, the markers exclude DIP2C. In other embodiments, the markers exclude GPM6B. In other embodiments, the markers exclude ITGAX. In other embodiments, the markers exclude LOC284412. In other embodiments, the markers exclude LOC285375. In other embodiments, the markers exclude MAMLD1. In other embodiments, the markers exclude MGC16121. In other embodiments, the markers exclude NDUFA10. In other embodiments, the markers exclude one or more of PDE9A. In other embodiments, the markers exclude PPAPDC1A. In other embodiments, the markers exclude PTPRN2. In other embodiments, the markers exclude RAP1GAP2. In other embodiments, the markers exclude RPS4Y2. In other embodiments, the markers exclude TUBA3D. In other embodiments, the markers exclude UBTD1.

As is understood by one of ordinary skill in the art, various methods of statistical analysis can be employed to identify cytosine methylation loci for use in the systems and methods disclosed herein.

In one embodiment, logistic regression analysis is used. Logistic regression analysis can lead to identification of the significant independent predictors among a number of possible predictors (e.g. methylation loci) known to be associated with increased risk of autism. Cytosine methylation levels at different loci can be used by themselves or in combination with other known risk predictors such as prenatal exposure to toxins (e.g. alcohol or maternal smoking, maternal diabetes, and family history). The probability that a subject has autism can be derived from the probability equation based on the logistic regression:

$${P}_{autism} = \frac{1}{1 + {e}^{- ( {α}_{1} {x}_{1} + {α}_{2} {x}_{2} + {α}_{3} {x}_{3} + … + {α}_{n} {x}_{n} )}}$$

where x refers to the magnitude or quantity of a particular predictor (e.g. methylation level at a particular locus) and a refers to the magnitude of change in the probability of the outcome (autism) for each unit change in the level of the particular predictor (x). The a values are derived from multivariable logistic regression analysis in a large population of affected and unaffected subjects. Values for x₁, x₂, x₃, . . . , xₙ, representing in this instance methylation percentage at different cytosine locI are derived from the subject being tested. Based on these values, a subject's probability of having a type of autism can be quantitatively estimated. Probability thresholds are used to define a high risk or low risk of autism. For example, if Pₐᵤₜᵢₛₘ≥1/100, the subject may be identified as being at high risk for autism, which may trigger further evaluation using, for example, any one or more of the following: CARS, ADOS-2, GARS-2, and ADI-R). Conversely, if Pₐᵤₜᵢₛₘ<1/200 or Pₐᵤₜᵢₛₘ<1/300, the subject may be identified as being at low risk for autism, and would require no further follow-up. The thresholds used can be based on the diagnostic sensitivity (number of autism cases correctly identified) and specificity (number of non-autism cases correctly identified as normal), as well as other factors considered clinically desirable, balanced by the risk and the medical cost of further interventions, such as assessments (psychological and otherwise) related to a designation of a subject as being at “high risk” for autism. Logistic regression analysis is well known as a method in disease screening for estimating a subject's risk for having a disorder. (Royston & Thompson, Stat. Med. 1992; 11:257-68.)

In another embodiment, a subject's risk of autism can also be calculated by using methylation percentages (reported as β-coefficients) at the individual discriminating cytosine locus by themselves or using different combinations of loci based on the method of overlapping Gaussian distribution or multivariate Gaussian distribution where the variable would be methylation level/percentage methylation at a particular (or multiple) loci. (See Wald, et al., BMJ 1988, 297, 883-887). Alternatively if methylation percentages or β-coefficients are not normally distributed (i.e. non-Gaussian), normal Gaussian distribution would be achieved if necessary by logarithmic transformation of these percentages.

For example, two Gaussian distribution curves are derived for methylation at particular loci in the autism and the normal populations. Mean, standard deviation (SD) and the degree of overlap between the two curves are then calculated. The ratio of the heights of the distribution curves at a given level of methylation will give the likelihood ratio or factor by which the risk of having autism is increased (or decreased) at a particular level of methylation at a given locus. The likelihood ratio (LR) value can be multiplied by the background risk of autism (or for a particular type of autism) in the general population and thus give a subject's risk of autism based on methylation level at the CG site(s) chosen. Information on the background population risk of autism in the newborn population is available from several sources. (See, for example, Hoffman, et al., Am. Heart J. 2004; 147:425-439). Similar information is available for prenatal and later postnatal life.

In a further embodiment, evolutionary computing can be used. Evolutionary computation methods are tools for predicting outcomes from a complex, large volume of data. Evolutionary computation includes a number of approaches such as genetic algorithms. This is widely utilized for problem solving and uses the three principles of natural evolution: selection, mutation, and recombination. (Penza-Reyes, et al., Artif. Intell. Med. 2000; 19:1-23; Whitley, Info Software Tech 2001; 43:87-31). Applications extend from chemistry, economics, engineering, and pharmaceuticals to metabolomics. The acute challenge of analyzing the vast volumes of data generated from new analytic platforms such as metabolomics has been outlined. (Goodcare, J. Exp. Bot. 2005; 56:245-54). As an example, the analysis of 250 biochemical markers (a plausible number of data points per subject in epigenetic analysis) was used to discriminate plants resistant to drought from normal control plants. A complete search to determine whether or not a particular metabolite would be included in the model would require 2²⁵⁰ or 1.8×10⁷⁵ computations. An ultrafast computer would require more than an estimated 3×10⁶² years to perform the required computations. Evolutionary computation is an automated method for providing a good solution for predicting the outcome from a large mass of data in a much shorter time.

Evolutionary computation selects ‘chromosomes’ (which is a string or a combination of different metabolites and their concentrations) that are optimally suited to ‘survive’ (i.e., predict the outcome of interest). Each predictor variable (e.g. metabolite) represents a ‘gene’ on this ‘chromosome’ string. The fitness to survive of each chromosome is a numerical value from 0 to 1, assigned by the computer program. ‘Fitness’ indicates how well this combination of parameters ensures evolutionary survival. (Goodcare, J. Exp. Bot. 2005, 56:245-54).

The combination of the ‘chromosome’ and the ‘fitness’ represents an ‘individual’. (Miranda, et al., Elec. Power Energ. Sys. 1998, 20:89-98). A population of such ‘individuals’ represents the ‘first generation’ of the organisms. The ‘individuals’ are ranked according to their fitness. This begins the evolutionary process. The selection operator creates the next generation by choosing the fittest individuals from the first generation which have the best chance of ‘survival’ i.e. predicting the outcome of interest. New second generation individuals are created by crossover with random rearrangement of segments of the ‘chromosome’ i.e. a change in a ‘chromosome’ segment with its string of constituent predictors (metabolite biomarkers) which form the sequence of ‘genes’. Finally, ‘mutation’ is produced where changes are introduced in an individual. Mutations could be either changes in constituent predictors or input variables (metabolite markers) with or without any change in their numerical values (concentrations).

Thus genetic algorithms take high performing ‘individuals’ and selects, ‘mutates’ and ‘recombines’ them with other high fitness or high performing ‘individuals’ to eventually achieve the optimal combination of ‘genes’ or input predictors on the ‘chromosome’ that will predict the outcome of interest. The similarities to the well-recognized principles of evolution are apparent. Evolutionary computing, including genetic algorithms, produces progressively better solutions to the problem through continuous reevaluation and adjustment. (Penza-Reyes & Sipper, Artif. Intell. Med. 2000, 19:1-23). The process identifies key components, and patterns form a large data set to achieve the highest predictive accuracy. The process is rapid, automated, and does not require any statistical or other assumptions about the input variables or outcomes of interest. It is unaffected by missing data, impervious to background noise, and does not require parametric distribution. Overall it is said to be superior to regression analyses and neural networks and equally handles both small and extremely large data sets. Given the large number of methylation sites analyzed, 450,000/subject DNA sample and the relatively small number of cases of autism, Genetic Programming, a branch of evolutionary computing, was the primary method of data analysis. The Gmax computer program version 11.09.23 (www.thegmax.com) was used for evolutionary computing analysis.

In additional embodiments when more than one marker is assayed, values of the detected markers can be calculated into a score. Each value can be weighted evenly within an algorithm generating a score, or the values for particular markers can be weighted more heavily in reaching the score. For example, markers with higher sensitivity and/or specificity scores could be weighted more heavily than markers with lower sensitivity and/or specificity scores. For example, marker values for diagnosing autism may be weighted as follows (i) (from highest weight to lowest weight): BCOR; PTPRN2; PDE9A; TUBA3D; LOC284412; (ii) (from highest weight to lowest weight): PDE9A; LOC284412; GPM6B; NDUFA10; (iii) (from highest weight to lowest weight): RPS4Y2; BCOR; UBTD1; PPAPDC1A; LOC285375; ITGAX; MGC16121; PTPRN2; CLCN4; D1P2C; or (iv) (from highest weight to lowest weight): UBTD1; RAP1GAP2; MAMLD1; C8orf75.

Markers may also be grouped into classes, and each class given a weighted score. For example, marker values for diagnosing autism may be grouped into classes and weighted as follows (from highest weight to lowest weight): Class 1: BCOR; PDE9A; UBTD1; RPS4Y2; Class 2: PTPRN2; LOC284412; RAP1GAP2; PPAPDC1A; LOC285375; Class 3: TUBA3D; GPM6B; MAMLD1; ITGAX; MGC16121; and Class 4: NDUFA10; C8orf75; CLCN4; and D1P2C.

Particular embodiments also include the following groups of markers presented in Tables 1-4 with associated percent contribution margins.

TABLE 1
Class 1 Cytosine Markers for Predicting and/or Diagnosing Autism
and their relative contribution margin.

| Locus | Gene Symbol | Chromosome | Contribution Margin (%) |
|---|---|---|---|
| cg03161453 | BCOR | X | 40 |
| cg15935227 | PTPRN2 | 7 | 20 |
| cg21639922 | PDE9A | 21 | 20 |
| cg18757828 | TUBA3D | 2 | 13.33 |
| cg04227007 | LOC284412 | 19 | 6.67 |

TABLE 2
Class 2 Cytosine Markers for Predicting and/or Diagnosing Autism
and their relative contribution margin.

| Locus | Gene Symbol | Chromosome | Contribution Margin (%) |
|---|---|---|---|
| cg21639922 | PDE9A | 21 | 38.24 |
| cg04227007 | LOC284412 | 19 | 23.53 |
| cg10479459 | GPM6B | X | 20.59 |
| cg05023192 | NDUFA10 | 2 | 17.65 |

TABLE 3
Class 3 Cytosine Markers for Predicting and/or Diagnosing Autism
and their relative contribution margin.

| Locus | Gene Symbol | Chromosome | Contribution Margin (%) |
|---|---|---|---|
| cg17741448 | RPS4Y2 | Y | 14.63 |
| cg04751297 | BCOR | X | 12.2 |
| cg10989317 | UBTD1 | 10 | 12.2 |
| cg10954330 | PPAPDC1A | 10 | 12.2 |
| cg20187719 | LOC285375 | 3 | 9.76 |
| cg04845171 | ITGAX | 16 | 9.76 |
| cg07283407 | MGC16121 | X | 9.26 |
| cg05175762 | PTPRN2 | 7 | 7.32 |
| cg00140189 | CLCN4 | X | 7.32 |
| cg01244571 | D1P2C | 10 | 4.88 |

TABLE 4
Class 4 Cytosine Markers for Predicting and/or Diagnosing Autism
and their relative contribution margin.

| Locus | Gene Symbol | Chromosome | Contribution Margin (%) |
|---|---|---|---|
| cg10989317 | UBTD1 | 10 | 48.78 |
| cg05932517 | RAP1GAP2 | 17 | 36.59 |
| cg16440909 | MAMLD1 | X | 12.2 |
| cg21297996 | C8orf75 | 8 | 2.44 |

Any marker or class of markers can be included in a particular value calculation. For example, in particular embodiments, Class 4 is included. In particular embodiments, Class 3 is included. In particular embodiments, Class 2 is included. In particular embodiments, Class 1 is included. In further embodiments, groups of classes can be included, for example, Classes 1 and 4; 1 and 3; 1 and 2; 4 and 3; 4 and 2; 3 and 2; etc.

Up- or down-regulation of the markers, as indicated elsewhere herein, for particular markers can be assessed by comparing a value to a relevant reference level. For example, the quantity of one or more markers can be indicated as a value. The value can be one or more numerical values resulting from the assaying of a sample, and can be derived, e.g., by measuring level(s) of the marker(s) in the sample by an assay, or from a dataset obtained from a provider such as a laboratory, or from a dataset stored on a server.

In the broadest sense, the value may be qualitative or quantitative. As such, where detection is qualitative, the systems and methods provide a reading or evaluation, e.g., assessment, of whether or not the marker is present in the sample being assayed. In further embodiments, the systems and methods provide a quantitative detection of whether the marker is present in the sample being assayed, i.e., an evaluation or assessment of the actual amount or relative abundance of the marker in the sample being assayed. In such embodiments, the quantitative detection may be absolute or relative, if the method is a method of detecting two or more different markers in a sample. As such, the term “quantifying” when used in the context of quantifying a marker in a sample can refer to absolute or to relative quantification. Absolute quantification can be accomplished by inclusion of known concentration(s) of one or more control markers and referencing, e.g., normalizing, the detected level of the marker with the known control markers (e.g., through generation of a standard curve). Alternatively, relative quantification can be accomplished by comparison of detected levels or amounts between two or more different markers to provide a relative quantification of each of the two or more markers, e.g., relative to each other. The actual measurement of values of the markers can be determined at the protein or nucleic acid level using any method known in the art.

As stated previously, obtained marker values can be compared to one or more reference levels. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from sample(s) and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of condition, etc.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than 0.05.

In particular embodiments, values obtained based on the markers and/or other dataset components can be subjected to an analytic process with chosen parameters. The parameters of the analytic process may be those disclosed herein or those derived using the guidelines described herein. The analytic process used to generate a result may be any type of process capable of providing a result useful for classifying a sample, for example, comparison of the obtained value with a reference level, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher.

The receiver operating characteristics (ROC) curve is a graph plotting sensitivity, which is defined in this setting as the percentage of autism cases with a positive test or abnormal cytosine methylation levels at a particular cytosine locus on the Y axis and false positive rate (1-specificity), i.e. the number of normal non-autism cases with abnormal cytosine methylation at the same locus on the X-axis. Specificity is defined as the percentage of normal cases with normal methylation levels at the locus of interest or a negative test. False positive rate refers to the percentage of normal subjects falsely found to have a positive test (i.e. abnormal methylation levels).

The area under the ROC curves (AUC) indicates the accuracy of the test in identifying normal from abnormal cases (Hanley & McNeil, Radiology 1982; 143:29-36).The AUC is the area under the ROC plot from the curve to the diagonal line from the point of intersection of the X- and Y-axes and with an angle of incline of 45°. The higher the area under the receiver operating characteristics (ROC) curve, the greater the accuracy of the test in predicting the condition of interest. An area ROC=1.0 indicates a perfect test, which is positive in all cases with the disorder and negative in all normal cases without the disorder.

In various embodiments, the values can be measured using methylation assays. “Methylation assay” refers to an assay, a large number of which are commercially available, for distinguishing methylated versus unmethylated cytosine loci in DNA. Commonly used techniques for measuring cytosine methylation include bisulfite-based methylation assays. The addition of bisulfite to DNA results in the methylation of the cytosine (i.e. addition of an extra carbon atom to position #5 of the hexagonal ring structure of the cytosine nucleotide) and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

Quantitative methylation assays include combined bisulfite and restriction analysis COBRA, which uses methylation sensitive restriction endonuclease, gel electrophoresis, and detection based on labeled hybridization probes. (Ziong and Laird, Nucleic Acid Res. 1997 25; 2532-4). Another exemplary assay is the methylation specific polymerase chain reaction PCR (MSPCR) for amplification of DNA segments of interest. This assay is performed after sodium bisulfite conversion of cytosine and uses methylation sensitive probes. Other methods include the Quantitative Methylation (QM) assay, which combines PCR amplification with fluorescent probes designed to bind to putative methylation sites; MethyLight™ (Qiagen, Redwood City, Calif.) a quantitative methylation assay that uses fluorescence based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are used to amplify the target sequence of interest. The amplified PCR product is isolated and used to quantitate the methylation status of the CpG site of interest. (Gonzalgo and Jones Nuclei Acids Res 1997; 25:252-31).

In specific embodiments, the ILLUMINA INFINIUM® (flumina, Inc., San Diego Calif., USA) Human Methylation 450 Beadchip assay is used. The Illumina assay can be used for genome wide quantitative methylation profiling. In various embodiments, genomic DNA can be extracted from cells, such as those from archived blood spot. Using techniques known to those of skill in the art, the genomic DNA can be isolated using commercial kits. Proteins and other contaminants can be removed from the DNA using proteinase K. The DNA can then be removed from the solution using available methods such as organic extraction, salting out, or binding the DNA to a solid phase support. As described above, and in the INFINIUM® Assay Methylation Protocol Guide, the DNA can be treated with sodium bisulfite, which converts unmethylated cytosine to uracil, while the methylated cytosine remains unchanged. The bisulfite converted DNA can then be denatured and neutralized. The denatured DNA can then be amplified. The next step uses enzymatic means to fragment the DNA. The fragmented DNA can then be precipitated using isopropanol and separated by centrifugation. The separated DNA can next be suspended in a hybridization buffer. The fragmented DNA can then be hybridized to beads that have been covalently limited to 50mer nucleotide segments at a locus specific to the cytosine nucleotide of interest in the genome. There are a total of over 500,000 bead types specifically designed to anneal to the locus where the particular cytosine is located. The beads are bound to silicon based arrays. There are two bead types designed for each locus, one bead type represents a probe that is designed to match to the methylated locus at which the cytosine nucleotide will remain unchanged. The other bead type corresponds to an initially unmethylated cytosine, which after sodium bisulfite treatment, is converted to uracil and ultimately a thiamine nuleotide. Unhybridized DNA (DNA not annealed to the beads) is washed away leaving only DNA segments bound to the appropriate bead and containing the cytosine of interest. If the cytosine of interest was unmethylated prior to the sodium bisulfite treatment, then it will match with the unmethylated or “U” bead probe. This enables single base extensions with fluorescent labeled nucleotide probes and generate fluorescent signals for that bead probe that can be read in an automated fashion. If the cytosine is methylated, single base mismatch will occur with the “U” bead probe oligomer. No further nucleotide extension on the bead oligomer occurs, thus preventing incorporation of the fluorescent tagged nucleotides on the bead. This will lead to low fluorescent signal from the “U” bead. The reverse will happen on the “M” or methylated bead probe.

Lasers are then used to stimulate the fluorophore bound to the single-base used for the sequence extension. The level of methylation at each cytosine locus is determined by the intensity of the fluorescence from the methylated compared to the unmethylated bead. Cytosine methylation level is expressed as “β” which is the ratio of the methylated-bead probe signal to total signal intensity at that cytosine locus. These techniques for determining cytosine methylation have been previously described and are widely available for commercial use.

Reliable identification of specific cytosine loci distributed throughout the genome has been detailed in the document “CpG Loci Identification. A guide to Ilumina's method for unambiguous CpG loci identification and tracking for the GOLDENGATE® and Infinium™ assays for Methylation”. (www.illumnia.com). Briefly, Illumina has developed a CpG locus identifier that designates cytosine loci based on the actual or contextual sequence of nucleotides in which the cytosine is located. It uses a similar strategy as used by NCBI's re SNP IPS (rs#) and is based on the sequence flanking the cytosine of interest. Thus a unique CpG locus cluster ID number is assigned to each of the cytosine undergoing evaluation. The system is reported to be consistent and will not be affected by changes in public databases and genome assemblies. Flanking sequences of 60 bases 5′ and 3′ to the CG locus (i.e. a total of 122 base sequences) is used to identify the locus. Thus a unique “CpG cluster number” or cg# is assigned to the sequence of 122 bp which contains the CpG of interest. Thus, only if the 122 bp in the CpG cluster is identical is there a risk of a locus being assigned the same number and being located in more than one position in the genome. Three separate criteria are utilized to track individual CpG locus based on this unique ID system, chromosome number, genomic coordinate, and genome build. The lesser of the two coordinates “C” or “G” in CpG is used in the unique CG loci identification. The CG locus is also designated in relation to the first ‘unambiguous” pair of nucleotides containing either an ‘A’ or ‘T’. If one of these nucleotides is 5′ to the CG then the arrangement is designated TOP and if such a nucleotide is 3′ it is designate BOT.

In addition, the forward or reverse DNA strand is indicated as being the location of the cytosine being evaluated. The assumption is made that methylation status of cytosine bases within the specific chromosome region is synchronized (Eckhart, et al., Nat. Gent. 2006, 38:1379-85).

Described herein are methods of using methylation technique to cover up to 99% of Ref Seq genes involving 16,000 genes and 500,000 cytosine nucleotides down to the single nucleotide level, throughout the genome. In various methods, the frequency of cytosine methylation of single nucleotides in a group of autism cases compared to controls is used to estimate the risk or probability of autism. The cytosine nucleotides analyzed using this technique included cytosines within CpG islands and those at further distances outside of the CpG islands i.e. located in “CpG shores” and “CpG shelves” and even more distantly located from the island so called “seas”.

DNA methylation is associated with altered gene expression and protein expression. It has been shown that DNA methylation leads to gene silencing. (Phillips et al., Nature Education 2008; 1(1):116; Hunter et al., Investigative Ophthalmology & Visual Science 2012; 53(4):2089).

Also described herein are methods of predicting autism and/or diagnosing autism using the measurement of mRNA levels transcribed by genes with altered cytosine methylation. Abnormal expression of mRNA transcribed from differentially methylated CG sites in genes and/or DNA sequences can also be used to predict and/or diagnose autism. In various embodiments, the measurement of RNA from related genes or genomic sequence levels using cells, tissues, and/or body fluids of subjects can be used to predict and/or diagnose autism. Any of the currently available techniques for determining expression levels of mRNA including Northern blot analysis, fluorescent in situ hybridization (FISH), RNase protection assays (RPA), microarrays, PCR-based, or other technologies for measuring RNA levels can be used.

Additionally, protein products of genes that are up- or down-regulated can be measured to assess cytosine methylation levels. Proteins translated from mRNA reflect the same phenomenon of altered gene function related to changes in cytosine methylation. Therefore, protein expression could also be used for the prediction and/or diagnosis of autism.

“Up-regulation” or “up-regulated” refers to an increase in the presence of a protein and/or an increase in the expression of the related gene. “Down-regulation” or “down-regulated” refers to a decrease in the presence of a protein and/or a decrease in the expression of the related gene. The “related gene” in reference to a particular protein refers to a nucleic acid sequence (used interchangeably with polynucleotide or nucleotide sequence) that encodes the particular protein. This definition also includes various sequence polymorphisms, mutations, and/or sequence variants wherein such alterations do not substantially affect the identity or function of the particular protein. For example, in a sequence identity analysis, the protein would share at least 80% sequence identity; at least 81% sequence identity; at least 82% sequence identity; at least 83% sequence identity; at least 84% sequence identity; at least 85% sequence identity; at least 86% sequence identity; at least 87% sequence identity; at least 88% sequence identity; at least 89% sequence identity; at least 90% sequence identity; at least 91% sequence identity; at least 92% sequence identity; at least 93% sequence identity; at least 94% sequence identity; at least 95% sequence identity; at least 96% sequence identity; at least 97% sequence identity; at least 98% sequence identity or at least 99% sequence identity with the particular protein.

“Protein detection” includes detection of full-length proteins, mature proteins, pre-proteins, polypeptides, isoforms, mutations, post-translationally modified proteins and variants thereof, and can be detected in any suitable manner.

In some embodiments, a marker is detected by contacting a sample with reagents (e.g., antibodies or nucleic acid primers), generating complexes of reagent and marker(s), and detecting the complexes. In various embodiments, measurement of the various proteins coded for by genes undergoing differential activation based on the differences in cytosine methylation can be used for the prediction and/or diagnosis of autism. Increased or decreased concentrations of proteins would result from changes in gene expression. Various methods for detecting and measuring protein levels can be used. These include western blot, immunohistochemistry, immunodiffusion, immunoassay, immunochemical, mass-spectrometry, immunoelectrophoresis, agglutination, and complement assays. Those skilled in the art will be familiar with numerous specific immunoassay formats and variations thereof which can be useful for carrying out the methods disclosed herein. See, e.g., E. Maggio, Enzyme-Immunoassay (1980), CRC Press, Inc., Boca Raton, Fla.; and U.S. Pat. Nos. 4,727,022; 4,659,678; 4,376,110; 4,275,149; 4,233,402; and 4,230,797. Examples of suitable immunoassays include immunoblotting, immunoprecipitation, immunofluorescence, chemiluminescence, electro-chemiluminescence (ECL), and/or enzyme-linked immunoassays (ELISA).

Antibodies can be conjugated to a solid support suitable for a diagnostic assay (e.g., beads such as protein A or protein G agarose, microspheres, plates, slides, or wells formed from materials such as latex or polystyrene) in accordance with known techniques, such as passive binding. Antibodies can be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques. Further, using techniques known to those of skill in the art, antibodies to epitopes in the protein(s) of interests can be developed for the purpose of detection of the protein(s).

Antibodies may also be useful for detecting post-translational modifications of markers. Examples of post-translational modifications include tyrosine phosphorylation, threonine phosphorylation, serine phosphorylation, citrullination and glycosylation (e.g., O-GlcNAc). Such antibodies specifically detect the phosphorylated amino acids in marker proteins of interest. These antibodies are well-known to those skilled in the art, and commercially available. Post-translational modifications can also be determined using metastable ions in reflector matrix-assisted laser desorption ionization-time of flight mass spectrometry (MALDI-TOF). See U. Wirth, et al., Proteomics 2002, 2(10):1445-1451.

Up- or down-regulation of genes also can be detected using, for example, cDNA arrays, cDNA fragment fingerprinting, cDNA sequencing, clone hybridization, differential display, differential screening, FRET detection, liquid microarrays, PCR, RT-PCR, quantitative RT-PCR analysis with TaqMan assays, molecular beacons, microelectric arrays, oligonucleotide arrays, polynucleotide arrays, serial analysis of gene expression (SAGE), and/or subtractive hybridization.

The term “gene” can include not only coding sequences but also regulatory regions such as promoters, enhancers, and termination regions. The term further can include all introns and other DNA sequences spliced from the mRNA transcript, along with variants resulting from alternative splice sites. Gene sequences encoding the particular protein can be nucleic acid sequences that direct the expression of the particular protein. These nucleic acid sequences may be a DNA strand sequence that is transcribed into RNA or an RNA sequence that is translated into the particular protein. The nucleic acid sequences include both the full-length nucleic acid sequences as well as non-full-length sequences encoding the full-length protein. The sequences can also include degenerate codons of the native sequence. Portions of complete gene sequences are referenced throughout the disclosure as is understood by one of ordinary skill in the art.

As an example, Northern hybridization analysis using probes that specifically recognize one or more marker sequences can be used to determine gene expression. Alternatively, expression can be measured using RT-PCR; e.g., polynucleotide primers specific for the differentially expressed marker mRNA sequences reverse-transcribe the mRNA into DNA, which is then amplified in PCR and can be visualized and quantified. Marker RNA can also be quantified using, for example, other target amplification methods, such as transcription-mediated amplification (TMA), strand displacement amplification (SDA), and Nucleic acid sequence based amplification (NASBA), or signal amplification methods (e.g., bDNA), and the like. Ribonuclease protection assays can also be used, using probes that specifically recognize one or more marker mRNA sequences, to determine gene expression.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Proteins and nucleic acids can be linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to proteins or nucleic acids on microarrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments).

Numerous protein and gene sequence markers are disclosed herein. The disclosure is not limited to the particularly disclosed protein and gene sequences but instead also encompasses sequences including 80% sequence identity; 81% sequence identity; 82% sequence identity; 83% sequence identity; 84% sequence identity; 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity or 99% sequence identity.

“% sequence identity” refers to a relationship between two or more sequences, as determined by comparing the sequences. In the art, “identity” also means the degree of sequence relatedness between protein (or nucleic acid) sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity can be found in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” mean any set of values or parameters which originally load with the software when first initialized.

Embodiments disclosed herein can be used with high throughput screening (HTS). Typically, HTS refers to a format that performs at least 100 assays, at least 500 assays, at least 1000 assays, at least 5000 assays, at least 10,000 assays, or more per day. When enumerating assays, either the number of samples or the number of protein or nucleic acid markers assayed can be considered.

Generally HTS methods involve a logical or physical array of either the subject samples, or the protein or nucleic acid markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, Mass.); Air Technical Industries (Mentor, Ohio); Beckman Instruments, Inc. (Fullerton, Calif.); Precision Systems, Inc. (Natick, Mass.), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

The systems and methods disclosed herein include kits. Disclosed kits include materials and reagents necessary to assay a sample obtained from a subject for one or more markers disclosed herein. The materials and reagents can include those necessary to assay the markers disclosed herein according to any method described herein and/or known to one of ordinary skill in the art.

Various embodiments include materials and reagents necessary to perform methylation assays on particular gene loci. Particular embodiments include materials and reagents necessary to assay for up- or down-regulation of a marker protein in a sample. In particular embodiments, the kits include antibodies to marker proteins and/or can also include aptamers, epitopes, or mimitopes. Other embodiments additionally or alternatively include oligonucleotides that specifically assay for one or more marker nucleic acids based on homology and/or complementarity with marker nucleic acids. The oligonucleotide sequences may correspond to fragments of the marker nucleic acids. For example, the oligonucleotides can be more than 200, 175, 150, 100, 50, 25, 10, or fewer than 10 nucleotides in length. Collectively, any molecule (e.g., antibody, aptamer, epitope, mimitope, oligonucleotide) that forms a complex with a marker is referred to as a marker binding agent herein.

Embodiments of kits can contain in separate containers marker binding agents either bound to a matrix, or packaged separately with reagents for binding to a matrix. In particular embodiments, the matrix is, for example, a porous strip. In some embodiments, measurement or detection regions of the porous strip can include a plurality of sites containing marker binding agents. In some embodiments, the porous strip can also contain sites for negative and/or positive controls. Alternatively, control sites can be located on a separate strip from the porous strip. Optionally, the different detection sites can contain different amounts of marker binding agents, e.g., a higher amount in the first detection site and lesser amounts in subsequent sites. Upon the addition of test sample, the number of sites displaying a detectable signal provides a quantitative indication of the amount of marker present in the sample. The detection sites can be configured in any suitably detectable shape and can be, e.g., in the shape of a bar or dot spanning the width (or a portion thereof) of a porous strip.

In some embodiments the matrix can be a solid substrate, such as a “chip.” See, e.g., U.S. Pat. No. 5,744,305. In some embodiments the matrix can be a solution array; e.g., xMAP (Luminex, Austin, Tex.), Cyvera (Illumina, San Diego, Calif.), RayBio Antibody Arrays (RayBiotech, Inc., Norcross, Ga.), CellCard (Vitra Bioscience, Mountain View, Calif.) and Quantum Dots' Mosaic (Invitrogen, Carlsbad, Calif.).

Additional embodiments can include control formulations (positive and/or negative), and/or one or more detectable labels, such as fluorescein, green fluorescent protein, rhodamine, cyanine dyes, Alexa dyes, luciferase, and radiolabels, among others. Instructions for carrying out the assay, including, optionally, instructions for generating a score, can be included in the kit; e.g., written, tape, VCR, or CD-ROM.

In particular embodiments, the kits include materials and reagents necessary to conduct and immunoassay (e.g., ELISA). In particular embodiments, the kits include materials and reagents necessary to conduct hybridization assays (e.g., PCR). In particular embodiments, materials and reagents expressly exclude equipment (e.g., plate readers). In particular embodiments, kits can exclude materials and reagents commonly found in laboratory settings (pipettes; test tubes; distilled H₂O).

Subjects include humans and research animals with a relevant model of autism (e.g., Norway rat (Rattus norvegicus); house mouse (Mus musculus); mu opioid knockout mice; FMR1 knockout mice; deer mice; songbirds (e.g., zebra finch)). Embodiments include the use of genome-wide differences in cytosine methylation in DNA to screen for and determine risk or likelihood of autism at any life stage. These stages include embryonic (from conception to 8 weeks of gestation), fetal (from 8 weeks of gestation to birth), neonatal (first 28 days after birth), infancy (up to 1 year of age), childhood (up to 10 years of age), adolescence (11 to 21 years of age), and adulthood (>21 years of age).

The sample can be any appropriate biological sample obtained from the subject. Cells and DNA from any biological sample(s) containing DNA can be used as a sample. Samples used for testing can be obtained from living or dead tissue and also archeological or forensic specimens containing cells or tissues. Exemplary samples include: body fluids (e.g. blood, serum, saliva, genital secretions, urine, cerebrospinal fluid (CSF), amniotic fluid, tears, breath condensate), skin, hair follicles/roots, mucous membranes (e.g. buccal scrapings or scrapings from the tongue), internal body tissue, umbilical cord segment, umbilical cord blood, or placental tissue. Additionally, cfDNA from cells that have been destroyed, and which can be retrieved from any body fluids, can be used as a sample.

In various embodiments, the methods disclosed herein can be used to predict autism in a subject before behavioral symptoms appear. In other embodiments, the methods disclosed herein can be used to diagnose autism in a subject after behavioral symptoms appear. In other embodiments, the methods disclosed herein can be used in conjunction with behavioral testing, such as the CARS, ADOS-2, GARS-2, ABC, or ADI-R, in order to diagnose autism in a subject. In further embodiments, the methods disclosed herein can be used to confirm a diagnosis of autism in a subject. In still further embodiments, the methods disclosed herein can be used to classify a subject as in need of further evaluation using behavioral testing.

Particular embodiments disclosed herein include obtaining a sample from a subject suspected of having autism; performing a methylation assay on the sample; determining one or more values based on the assaying; comparing the one or more values to a reference level; and predicting or diagnosing autism in the subject according to the methylation status of a marker, as described elsewhere herein.

Particular embodiments also include predicting or diagnosing autism in a subject by obtaining a sample from a subject suspected of having autism; assaying the sample for up- or down-regulation of one or more markers disclosed herein; determining one or more marker values based on the assaying; comparing the one or more marker values to a reference level; and predicting or diagnosing autism in the subject according to the methylation status of a marker as determined by the up- or down-regulation of the one or more markers, as described elsewhere herein.

Various embodiments include obtaining a sample from a subject suspected of having autism; performing a methylation assay on the sample; determining one or more values based on the assaying; comparing the one or more values to a reference level; and predicting autism in the subject according to the methylation status of one or more markers, as described elsewhere herein.

Further embodiments include predicting autism in a subject by obtaining a sample from a subject suspected of having autism; assaying the sample for up- or down-regulation of one or more markers disclosed herein; determining one or more marker values based on the assaying; comparing the one or more marker values to a reference level; and predicting autism in the subject according to the methylation status of a marker as determined by the up- or down-regulation of the one or more markers, as described elsewhere herein.

Other embodiments include obtaining a sample from a subject suspected of having autism; performing a methylation assay on the sample; determining one or more values based on the assaying; comparing the one or more values to a reference level; and diagnosing autism in the subject according to the methylation status of one or more markers, as described elsewhere herein.

Additional embodiments include diagnosing autism in a subject by obtaining a sample from a subject suspected of having autism; assaying the sample for up- or down-regulation of one or more markers disclosed herein; determining one or more marker values based on the assaying; comparing the one or more marker values to a reference level; and diagnosing autism in the subject according to the methylation status of a marker as determined by the up- or down-regulation of the one or more markers, as described elsewhere herein.

Other embodiments include obtaining a sample from a subject suspected of having autism; performing a methylation assay on the sample; determining one or more values based on the assaying; comparing the one or more values to a reference level; performing behavioral testing on the subject; and diagnosing autism in the subject according to the methylation status of one or more markers, as described elsewhere herein, and the results of the behavioral testing.

Additional embodiments include diagnosing autism in a subject by obtaining a sample from a subject suspected of having autism; assaying the sample for up- or down-regulation of one or more markers disclosed herein; determining one or more marker values based on the assaying; comparing the one or more marker values to a reference level; performing behavioral testing on the subject; and diagnosing autism in the subject according to the methylation status of a marker as determined by the up- or down-regulation of the one or more markers, as described elsewhere herein, and the results of the behavioral testing.

Other embodiments include obtaining a sample from a subject diagnosed with autism; performing a methylation assay on the sample; determining one or more values based on the assaying; comparing the one or more values to a reference level; and confirming a diagnosis of autism in the subject according to the methylation status of one or more markers, as described elsewhere herein.

Additional embodiments include confirming a diagnosis of autism in a subject by obtaining a sample from a subject suspected of having autism; assaying the sample for up- or down-regulation of one or more markers disclosed herein; determining one or more marker values based on the assaying; comparing the one or more marker values to a reference level; and confirming an autism diagnosis in the subject according to the methylation status of a marker as determined by the up- or down-regulation of the one or more markers, as described elsewhere herein.

A prediction or diagnosis according to the systems and methods disclosed herein can direct a treatment regimen. For example, an autism diagnosis can direct treatment with an autism treatment (e.g., lifestyle and behavioral interventions; behavioral management therapy; cognitive behavior therapy; early intervention; educational and school-based therapies; joint attention therapy; medication treatment; nutritional therapy; occupational therapy; parent-mediated therapy; physical therapy; social skills training; speech-language therapy). Administered treatments will be delivered in therapeutically effective amounts leading to an improvement or resolution of the treated condition, as assessed by a practicing physician or researcher.

The present disclosure provides kits for diagnosing autism in a subject. The kit can include components for detecting, identifying, and/or quantitating cytosine methylation of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the following genes encoding BCL6 co-repressor (BCOR); long intergenic non-protein coding RNA 589 (C8orf75); chloride channel, voltage-sensitive chloride channel 1, (CLCN1); chloride channel voltage-sensitive 4 (CLCN4); disco-interacting protein 2 homolog C (D1P2C); glycoprotein M6B (GPM6B); integrin, alpha X complement component 3 receptor 4 subunit (ITGAX); LOC284412; long intergenic non-protein coding RNA 620 (LOC285375); mastermind-like domain containing 1 (MAMLD1); MIR503 host gene (MGC16121); NADH dehydrogenase (ubiquinone) 1 alpha subcomplex, 10 (NDUFA10); phosphodiesterase 9A (PDE9A); phosphatidic acid phosphatase type 2 domain containing 1A (PPAPDC1A); protein tyrosine phosphatase, receptor type, N polypeptide 2 (PTPRN2); RAP1 GTPase activating protein 2 (RAP1GAP2); ribosomal protein S4, Y-linked 2 (RPS4Y2); tubulin, alpha 3d (TUBA3D); and ubiquitin domain containing 1 (UBTD1). The kit can also include components for detecting and/or quantitating expression of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the following genes encoding BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The kit can also include components for detecting and/or quantitating expression of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the following proteins: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The kits can comprise a microarray including one or more of the genes or proteins for diagnosing autism.

The present disclosure provides microarray for diagnosing autism by detecting and/or quantitating the expression of one or more of the following genes encoding BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The microarray can include nucleic acids that bind to one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the following genes encoding BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The microarray can include one to eighteen different nucleic acids, each binding to one of the following genes: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1.

The present disclosure also provides microarray for diagnosing autism by detecting and/or quantitating the expression of one or more of the following proteins: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The microarray can include antibodies or proteins that bind to one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the following proteins: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1. The microarray can include one to eighteen different antibodies or proteins, each binding to one of the following proteins: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, and UBTD1.

Binding to proteins or nucleic acids on microarrays can be detected by scanning the microarray with a variety of laser or CCD-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments).

Autism can be diagnosed by determining that there is altered gene expression or protein expression in a subject of a marker gene described herein or of a protein encoded by a marker gene described herein as compared to the corresponding gene or protein in a control sample or reference value. Austism also can be diagnosed by determining that there is an altered cytosine methylation located on one of the marker genes. The control sample or reference value can be obtained from a normal or unaffected subject.

A blood sample can be maternal blood obtained during the prenatal period from 11 weeks to 42 weeks of pregnancy. A blood sample can be obtained from a neonatal subject within 72 hours of birth or within 7 days of after birth, from a neonatal subject from 7 days after birth up to 28 days after birth, from an infant subject from 29 days after birth up to one year of age, or from a subject from one year of age up to 17 years of age. The blood sample can be analyzed for altered gene or protein expression or for altered methylation of cytosines of one or more of the marker genes.

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXAMPLES

### Example 1

Description of the Methods. A single neonatal dried blood spot saved on filter paper was retrieved from biobank specimens collected as part of the newborn screening program for the detection of metabolic disorders and stored by the Michigan Department of Community Health in Lansing, Mich. Blood was originally obtained by heel-stick and placed on filter paper an average of two days after birth. Samples were stored at room temperature. De-identified residual blood spots after the completion of clinical testing were used. Parental consent was obtained for the use of the residual blood spots. The Institutional Review Board approval was obtained through a standardized process. The specimens used for the current study were collected between 1998 and 2003. Cases with chromosomal abnormalities, other known or suspected genetic syndromes, or other significant medical or surgical disorders were excluded.

A total of 10 cases of and 14 unaffected controls were analyzed. Control cases were normal newborns with no significant medical or surgical disorder at the time of the blood draw.

DNA extraction from blood-spot. DNA extraction was performed as described in the EZ1® DNA Investigator Handbook, Sample and Assay Technologies, QUIAGEN 4th Edition, April 2009. Briefly, two 6 mm diameter circles (or four 3mm diameter circles) were punched out of a dried blood spot stored on filter paper and used for DNA extraction. The circle contains DNA from white blood cells from 5 μL of whole blood. The circles are transferred to a 2 ml sample tube.

A total of 190 μL of diluted buffer G2 (G2 buffer: distilled water in 1:1 ratio) was used to elute DNA from the filter paper. Additional buffer is added until residual sample volume in the tube is 190 μL because filter paper will absorb a certain volume of the buffer. Ten μL of proteinase K is added and the mixture is vortexed for 10 seconds and quick spun. The mixture is then incubated at 56° C. for 15 minutes at 900 rpm. Further incubation at 95° C. for 5 minutes at 900 rpm is performed to increase the yield of DNA from the filter paper. Quick spin was performed. The sample is then run on EZ1 Advanced (Trace, Tip-Dance) protocol as described. The protocol is designed for isolation of total DNA from the mixture. Elution tubes containing purified DNA in 50 μL of water is now available for further analysis.

Infinium DNA methylation assay. Illumina's Infinium Human Methylation 450 Bead Chip system was used for genome-wide methylation analysis. DNA (500 ng) was subjected to bisulfite conversion to deaminate unmethylated cytosines to uracils with the EZ-96 Methylation Kit (Zymo Research) using the standard protocol for Infinium. The DNA is enzymatically fragmented and hybridized to the Illumina BeadChips. BeadChips contain locus-specific oligomers and are in pairs, one specific for the methylated cytosine locus and the other for the unmethylated locus. A single base extension is performed to incorporate a biotin-labeled ddNTP. After fluorescent staining and washing, the BeadChip is scanned and the methylation status of each locus is determined using BeadStudio software (Illumina). Experimental quality was assessed using the Controls Dashboard that has sample-dependent and sample-independent controls target removal, staining, hybridization, extension, bisulfite conversion, specificity, negative control, and non-polymorphic control. The methylation status is the ratio of the methylated probe signal relative to the sum of methylated and unmethylated probes. The resulting ratio indicates whether a locus is unmethylated (0) or fully methylated (1). Differentially methylated sites are determined using the Illumina Custom Model and filtered according to p-value using 0.05 as a cutoff.

IIlumina's Infinium HumanMethylation 450 BeadChip system covers CpG sites in the promoter region of 16,880 genes. In addition, other cytosine loci throughout the genome and outside of genes, and within or outside of CpG islands, are represented in this assay.

Cytosine Methylation for the Prediction of Autism Risk Using ROC Curve. To determine the accuracy of the methylation level of a particular cytosine locus for autism prediction, different threshold levels of methylation at the site e.g. ≥10%, ≥20%, ≥30%, ≥40%; etc. were used to calculate sensitivity and specificity for autism prediction. Thus, for example, using ≥10% methylation at a particular CG locus, cases with methylation levels above this threshold would be considered to have a positive test and those with lower than this threshold are interpreted as a negative methylation test. The percentage of autism cases with a positive test in this example ≥10% methylation at this particular cytosine locus would be equal to the sensitivity of the test. The percentage of normal non-autism cases with cytosine methylation levels of <10% at this locus would be considered the specificity of the test. False positive rate is here defined as the number of normal cases with a (falsely) abnormal test result and sensitivity is defined as the number of autism cases with (correctly) abnormal test result i.e. the level of methylation ≥10% at this particular CG location. A series of threshold methylation values are evaluated e.g. ≥ 1/10, ≥ 1/20, ≥ 1/30 etc., and used to generate a series of paired sensitivity and false positive values for each locus. A receiver operating characteristic (ROC) curve which is a plot of data points with sensitivity values on the Y-axis and false positivity rate on the X-axis is generated. This approach can be used to generate ROC curves for each individual cytosine locus that displays significant methylation differences between cases and autism groups.

It should be noted that similarly, in loci at which the methylation level was reduced in affected compared to control cases threshold methylation values for calculating sensitivity and false positive rates would be ≥ 1/10, ≥ 1/20, ≥ 1/30, etc.

Standard statistical testing using p-values to express the probability that the observed difference between cytosine methylation at a given locus between autism and control DNA specimens were performed.

More stringent testing using False discovery Rate (FDR) was also performed. The FDR gives the probability that positive results were due to chance when multiple hypothesis testing is performed using multiple comparisons. The Benjamini-Hochberg test was used to calculate FDR.

### Example 2

Blood spots were collected on filter paper from newborns undergoing routine screening for metabolic disorders. Newborns averaged two days of age at the time of collection. Completely de-identified residual blood spots not used for metabolic testing was stored at room temperature at the Michigan Department of Community Health facilities in Lansing, Mich. DNA was extracted and purified from a single spot of blood on filter paper as described previously in the application and methylation levels in different CPG islands determined using the Illumina's Infinium Human Methylation 450 Bead Chip system.

The level or percentage methylation at multiple cytosine throughout the DNA was compared in 14 cases of autism versus 10 normal cases. Table 1 shows five cytosine loci located in five different genes that were associated with significant differences in methylation between autism cases and the normal controls. The GENE symbols, chromosome number on which the gene is located, and the specific cytosine locus displaying differential methylation are provided along with the contribution (‘marginal contribution’) of each particular cytosine locus for the overall prediction of autism versus normal controls. The extremely low FDR values of each cytosine locus (Table 5) indicate the highly significant differences in the percentage methylation between these specific cytosines in autism cases versus controls. The high diagnostic performance (AUC, sensitivity, specificity, and low p-values for AUC) of the cytosine biomarker combination in Table 1 are shown in Table 6. This model only considered cytosine biomarkers in autism prediction and used the minimum number of predictive sites in the model.

TABLE 5
Differentially Methylated Cytosine Loci and associated Genes in
Autism.

| | | | % m | | FDR | |
|---|---|---|---|---|---|---|
| TargetID | GeneSym | Chromosome | Change | pvalue | pvalue | AUC |
| cg00140189 | CLCN4 | X | 13.74038 | 3.68E−38 | 3.47E−35 | 0.91429 |
| cg00150874 | BCOR; BCOR | X | 14.81594 | 3.68E−38 | 3.47E−35 | 0.91429 |
| cg00308367 | KDM5D | Y | 38.21149 | 3.68E−38 | 3.47E−35 | 0.89286 |
| cg00408231 | ARMCX1 | X | 17.65587 | 3.68E−38 | 3.47E−35 | 0.88571 |
| cg00501169 | NBPF4 | 1 | 19.97094 | 3.68E−38 | 3.47E−35 | 0.87857 |
| cg00576139 | LOC100101115; | Y | 14.61021 | 3.68E−38 | 3.47E−35 | 0.87857 |
| | TTTY21 | | | | | |
| cg00632358 | SNX29 | 16 | 7.812852 | 3.68E−38 | 3.47E−35 | 0.87857 |
| cg00676506 | RPS4Y2 | Y | 15.69715 | 3.68E−38 | 3.47E−35 | 0.87857 |
| cg00756172 | LOC340094 | 5 | 13.13532 | 3.68E−38 | 3.47E−35 | 0.87857 |
| cg00944884 | NBL1 | 1 | 21.76583 | 3.68E−38 | 3.47E−35 | 0.87143 |
| cg01078565 | MAGEB2 | X | 11.20327 | 3.68E−38 | 3.47E−35 | 0.87143 |
| cg01127608 | LMX1B | 9 | 20.881 | 3.68E−38 | 3.47E−35 | 0.86429 |
| cg01153376 | MSLN; MIR662 | 16 | 14.76771 | 3.68E−38 | 3.47E−35 | 0.86429 |
| cg01244571 | DIP2C | 10 | 7.70281 | 3.68E−38 | 3.47E−35 | 0.86429 |
| cg01347786 | RBMS3; RBMS3 | 3 | 15.53262 | 3.68E−38 | 3.47E−35 | 0.86429 |
| cg01426558 | DDX3Y | Y | 23.00077 | 3.68E−38 | 3.47E−35 | 0.86429 |
| cg01451277 | DPP8 | 15 | 18.90162 | 3.68E−38 | 3.47E−35 | 0.86429 |
| cg01498999 | NLGN4Y | Y | 23.62679 | 3.68E−38 | 3.47E−35 | 0.85714 |
| cg01522249 | BCOR | X | 17.21601 | 3.68E−38 | 3.47E−35 | 0.85714 |
| cg01600516 | ALOX12 | 17 | 32.79307 | 3.68E−38 | 3.47E−35 | 0.85714 |
| cg01677142 | OCA2 | 15 | 13.84752 | 3.68E−38 | 3.47E−35 | 0.85714 |
| cg01900066 | EIF1AY | Y | 17.74769 | 3.68E−38 | 3.47E−35 | 0.85714 |
| cg01906946 | BCOR | X | 17.4637 | 3.68E−38 | 3.47E−35 | 0.85714 |
| cg01911472 | TBL1Y | Y | 19.86583 | 3.68E−38 | 3.47E−35 | 0.85714 |
| cg01958928 | TPD52 | 8 | 15.67355 | 3.68E−38 | 3.47E−35 | 0.85714 |
| cg01984154 | RPS4Y1 | Y | 35.63903 | 3.68E−38 | 3.47E−35 | 0.85 |
| cg02004401 | GNA12 | 7 | 17.42085 | 3.68E−38 | 3.47E−35 | 0.85 |
| cg02011394 | TSPY4 | Y | 26.04095 | 3.68E−38 | 3.47E−35 | 0.85 |
| cg02050847 | RPS4Y2 | Y | 38.41111 | 3.68E−38 | 3.47E−35 | 0.85 |
| cg02056550 | TTTY18 | Y | 31.49682 | 3.68E−38 | 3.47E−35 | 0.85 |
| cg02352633 | RBMY2EP | Y | 22.16595 | 3.68E−38 | 3.47E−35 | 0.85 |
| cg02394572 | AMZ1 | 7 | 39.39936 | 3.68E−38 | 3.47E−35 | 0.85 |
| cg02407581 | TTTY18 | Y | 22.48185 | 3.68E−38 | 3.47E−35 | 0.85 |
| cg02522936 | TTTY10 | Y | 26.68201 | 3.68E−38 | 3.47E−35 | 0.85 |
| cg02730008 | TMSB4Y | Y | 30.91268 | 3.68E−38 | 3.47E−35 | 0.84286 |
| cg02741327 | TMEM163 | 2 | 19.46717 | 3.68E−38 | 3.47E−35 | 0.84286 |
| cg02788633 | MICALL2 | 7 | 12.48116 | 3.68E−38 | 3.47E−35 | 0.84286 |
| cg02907689 | PDZD4 | X | 13.92036 | 3.68E−38 | 3.47E−35 | 0.84286 |
| cg02931660 | BCOR | X | 14.19786 | 3.68E−38 | 3.47E−35 | 0.83571 |
| cg03052502 | FAM197Y2 | Y | 33.82447 | 3.68E−38 | 3.47E−35 | 0.83571 |
| cg03161453 | BCOR | X | 19.98807 | 3.68E−38 | 3.47E−35 | 0.83571 |
| cg03183700 | ANK1 | 8 | 13.17744 | 3.68E−38 | 3.47E−35 | 0.83571 |
| cg03199239 | GNG7 | 19 | 22.89847 | 3.68E−38 | 3.47E−35 | 0.83571 |
| cg03278611 | NLGN4Y | Y | 35.17792 | 3.68E−38 | 3.47E−35 | 0.83571 |
| cg03293837 | ATE1 | 10 | 14.89882 | 3.68E−38 | 3.47E−35 | 0.83571 |
| cg03443143 | TSPY4; | Y | 21.90664 | 3.68E−38 | 3.47E−35 | 0.82857 |
| | FAM197Y2 | | | | | |
| cg03492634 | GP5 | 3 | 17.42065 | 3.68E−38 | 3.47E−35 | 0.82857 |
| cg03535417 | TSPY4; | Y | 21.02525 | 3.68E−38 | 3.47E−35 | 0.82857 |
| | FAM197Y2 | | | | | |
| cg03554089 | XIST | X | 20.81236 | 3.68E−38 | 3.47E−35 | 0.82857 |

TABLE 6
Cytosine Methylation Prediction of Autism: Evolutionary Computing.

| Biomarkers | AUC | Sensitivity | Specificity | P-value |
|---|---|---|---|---|
| Refer to Table 1 | 1.00 | 100.0 | 100.0 | <0.00001 |
| Refer to Table 2 | 1.00 | 100.0 | 100.0 | <0.00001 |
| Refer to Table 3 | 1.00 | 100.0 | 100.0 | <0.00001 |
| Refer to Table 4 | 1.00 | 100.0 | 100.0 | <0.00001 |

### Example 3

Blood spots were collected on filter paper from newborns undergoing routine screening for metabolic disorders. Newborns averaged two days of age at the time of collection. Completely de-identified residual blood spots not used for metabolic testing was stored at room temperature at the Michigan Department of Community Health facilities in Lansing, Mich. DNA was extracted and purified from a single spot of blood on filter paper as described previously and methylation levels in different CPG islands determined using the Illumina's Infinium Human Methylation 450 Bead Chip system.

Demographic and clinical factors have been shown in multiple studies to affect risk of development of autism. We therefore considered the potential impact of putative risk factors such as race, gender, gestational age at delivery, birth weight, and plausible factors such as interval between birth and blood draw and also length of storage of the blood specimens. It is possible that the latter two factors could affect DNA methylation. We therefore considered all the above demographic, clinical, and specimen handling factors with biomarkers for the prediction of autism.

The level or percentage methylation at multiple cytosine loci throughout the DNA was compared in 14 cases of autism versus 10 normal cases. Table 4 shows four cytosine loci located in known genes that were associated with significant differences in methylation between autism cases and the controls. The GENE symbols, chromosome number on which the gene is located, and the specific cytosine locus displaying differential methylation are provided along with the contribution (marginal contribution') of each particular cytosine locus for the overall prediction of autism versus normal controls. In this analysis, demographic and clinical characteristics such as maternal age and race, gestational age of delivery, birth weight, newborn gender, and interval from birth to sample collection were also considered as potential contributors to autism prediction. None of these factors significantly contributed to prediction. For the cytosine loci, the extremely low FDR values (Tables 5 and 7) indicate the highly significant differences in the percentage methylation between these specific cytosines in autism cases versus controls. The high diagnostic performance (AUC, sensitivity, specificity, and low p-values for AUC) are shown in Table 6. This model (“parsimonious”) used a minimum number of DNA methylation markers for prediction.

TABLE 7
Differentially Methylated Cytosine Loci and associated Genes in
Autism

| | Gene | | | | FDR p- | |
|---|---|---|---|---|---|---|
| TargetID | Sym | Chromosome | % m Change | pvalue | value | AUC |
| cg03554089 | XIST | X | 20.81236 | 3.68E−38 | 3.47E−35 | 0.7 |
| cg16995742 | COPS8 | 2 | 17.77194 | 3.68E−38 | 3.47E−35 | 0.7 |
| cg17869311 | GXYLT2 | 3 | 15.70243 | 3.68E−38 | 3.47E−35 | 0.7 |
| cg19278629 | LMNB2 | 19 | 17.39525 | 3.68E−38 | 3.47E−35 | 0.7 |
| cg20089799 | TSPAN9 | 12 | 22.71465 | 3.68E−38 | 3.47E−35 | 0.7 |
| cg21903981 | PAPLN | 14 | 20.47229 | 3.68E−38 | 3.47E−35 | 0.7 |
| cg00150874 | BCOR | X | 14.81594 | 3.68E−38 | 3.47E−35 | 0.69286 |
| cg02907689 | PDZD4 | X | 13.92036 | 3.68E−38 | 3.47E−35 | 0.69286 |
| cg09247979 | PTPRK | 6 | 25.84962 | 3.68E−38 | 3.47E−35 | 0.69286 |
| cg13183651 | KRTAP3-3 | 17 | 19.3143 | 3.68E−38 | 3.47E−35 | 0.69286 |
| cg18057692 | CAMKK1 | 17 | 14.27172 | 3.68E−38 | 3.47E−35 | 0.69286 |
| cg19248407 | CUX1 | 7 | 20.19702 | 3.68E−38 | 3.47E−35 | 0.69286 |
| cg19569170 | ARHGAP26 | 5 | 19.73967 | 3.68E−38 | 3.47E−35 | 0.69286 |
| cg00756172 | LOC340094 | 5 | 13.13532 | 3.68E−38 | 3.47E−35 | 0.68571 |
| cg04381873 | LOC284412 | 19 | 14.62235 | 3.68E−38 | 3.47E−35 | 0.68571 |
| cg04787784 | LMF1 | 16 | 22.28741 | 3.68E−38 | 3.47E−35 | 0.68571 |
| cg05292605 | LTBP2 | 14 | 17.17855 | 3.68E−38 | 3.47E−35 | 0.68571 |
| cg05341252 | HLA-DQB1 | 6 | 21.68212 | 3.68E−38 | 3.47E−35 | 0.68571 |
| cg10039267 | BCOR | X | 16.31939 | 3.68E−38 | 3.47E−35 | 0.68571 |
| cg11268327 | MICA | 6 | 20.32274 | 3.68E−38 | 3.47E−35 | 0.68571 |
| cg12208638 | ACTN3 | 11 | 23.52442 | 3.68E−38 | 3.47E−35 | 0.68571 |
| cg13176022 | XG | X | 14.83821 | 3.68E−38 | 3.47E−35 | 0.68571 |
| cg13966843 | C6orf10 | 6 | 12.05593 | 3.68E−38 | 3.47E−35 | 0.68571 |
| cg14261068 | BCOR | X | 17.88543 | 3.68E−38 | 3.47E−35 | 0.68571 |
| cg22508145 | CPAMD8 | 19 | 19.75364 | 3.68E−38 | 3.47E−35 | 0.68571 |
| cg02522936 | TTTY10 | Y | 26.68201 | 3.68E−38 | 3.47E−35 | 0.67857 |
| cg08477332 | S100A14 | 1 | 19.75707 | 3.68E−38 | 3.47E−35 | 0.67857 |
| cg09829904 | ZFY | Y | 24.34083 | 3.68E−38 | 3.47E−35 | 0.67857 |
| cg12012426 | KIAA1530 | 4 | 23.02595 | 3.68E−38 | 3.47E−35 | 0.67857 |
| cg21797452 | LOC286467 | X | 19.78103 | 3.68E−38 | 3.47E−35 | 0.67857 |
| cg22007216 | DCHS2 | 4 | 21.76253 | 3.68E−38 | 3.47E−35 | 0.67857 |
| cg22802014 | SNRNP40 | 1 | 17.01872 | 3.68E−38 | 3.47E−35 | 0.67857 |
| cg24156613 | BCOR | X | 18.64296 | 3.68E−38 | 3.47E−35 | 0.67857 |
| cg26570714 | OR2T10 | 1 | 19.76158 | 3.68E−38 | 3.47E−35 | 0.67857 |
| cg02056550 | TTTY18 | Y | 31.49682 | 3.68E−38 | 3.47E−35 | 0.67143 |
| cg02931660 | BCOR | X | 14.19786 | 3.68E−38 | 3.47E−35 | 0.67143 |
| cg04624564 | NCRNA00182 | X | 15.68838 | 3.68E−38 | 3.47E−35 | 0.67143 |
| cg05990366 | FAM101A | 12 | 12.53592 | 3.68E−38 | 3.47E−35 | 0.67143 |
| cg06084534 | OR6K6 | 1 | 18.94504 | 3.68E−38 | 3.47E−35 | 0.67143 |
| cg07876831 | TMCO3 | 13 | 24.80591 | 3.68E−38 | 3.47E−35 | 0.67143 |
| cg08225549 | STMN2 | 8 | 19.52434 | 3.68E−38 | 3.47E−35 | 0.67143 |
| cg15366127 | BCOR | X | 16.12798 | 3.68E−38 | 3.47E−35 | 0.67143 |
| cg15516537 | RBMY1D; | Y | 18.16238 | 3.68E−38 | 3.47E−35 | 0.67143 |
| | RBMY1A1; | | | | | |
| | RBMY1E; | | | | | |
| | RBMY1B | | | | | |
| cg19504860 | ZNF708 | 19 | 23.13524 | 3.68E−38 | 3.47E−35 | 0.67143 |
| cg27020349 | UTRN | 6 | 16.59896 | 3.68E−38 | 3.47E−35 | 0.67143 |
| cg08707617 | BCOR | X | 15.79325 | 3.68E−38 | 3.47E−35 | 0.66429 |
| cg10102162 | SORCS1 | 10 | 10.9724 | 3.68E−38 | 3.47E−35 | 0.66429 |
| cg10601372 | WDR18 | 19 | 10.16212 | 3.68E−38 | 3.47E−35 | 0.66429 |
| cg13284789 | SIDT1 | 3 | 26.2783 | 3.68E−38 | 3.47E−35 | 0.66429 |
| cg18001722 | GRK5 | 10 | 16.70102 | 3.7E−38 | 3.5E−35 | 0.66429 |
| cg19712277 | MMEL1 | 1 | 19.2261 | 3.7E−38 | 3.5E−35 | 0.66429 |
| cg25705492 | TSPY4 | Y | 22.52221 | 3.7E−38 | 3.5E−35 | 0.66429 |
| cg01127608 | LMX1B | 9 | 20.881 | 3.7E−38 | 3.5E−35 | 0.65714 |
| cg07240846 | CAMK1D | 10 | 17.33648 | 3.7E−38 | 3.5E−35 | 0.65714 |
| cg04173211 | IL16 | 15 | 16.44636 | 3.7E−38 | 3.5E−35 | 0.65 |
| cg06051619 | DIP2C | 10 | 19.4605 | 3.7E−38 | 3.5E−35 | 0.65 |
| cg06350542 | MCF2L | 13 | 15.39299 | 3.7E−38 | 3.5E−35 | 0.65 |
| cg07747963 | RPS4Y2 | Y | 24.22948 | 3.7E−38 | 3.5E−35 | 0.65 |
| cg11585022 | WDR36 | 5 | 18.95778 | 3.7E−38 | 3.5E−35 | 0.65 |
| cg11970733 | GLT1D1 | 12 | 15.31366 | 3.7E−38 | 3.5E−35 | 0.65 |
| cg12727358 | CACNA1D | 3 | 18.43226 | 3.7E−38 | 3.5E−35 | 0.65 |

The results presented herein confirm that based on the differences in the level of methylation of the cytosine sites between autism and normal cases throughout the whole human genome, the predisposition to or risk of having a autism can be determined.

Without being bound to theory, the differences in methylation can be explained by the development of autism resulting from or leading to abnormal expression of multiple genes many of which directly or indirectly impact or control neurodevelopment. Abnormal gene function includes either the suppression of the function of genes whose activities are important to normal brain development or conversely the activation of genes whose functions are normally suppressed to permit normal development of the brain. Thus, genome wide cytosine methylation study provides information on the orchestrated widespread activation and suppression of multiple genes and gene networks involved in the normal and abnormal development of the brain. The approach does not require prior knowledge of the role of particular genes in brain development or the mechanism by which changes in the function of the genes lead to autism. Further, hundreds of thousands of cytosine loci involving thousands of genes are evaluated simultaneously and in an unbiased fashion and can thus be used to accurately estimate the risk of autism. Of further importance is the fact that cytosine loci outside of the genes can also control gene function, so methylation levels of a large number of loci located outside of the gene further contribute to the prediction of autism.

It has been confirmed that aberration or change in the methylation pattern of cytosine nucleotide occur at multiple cytosine loci throughout the genome in subjects affected with development of autism compared to subjects without autism.

### Example 4

Introduction. Highly significant differences in the percentage of methylation of cytosine nucleotides throughout the genome in subjects with autism as compared to normal groups have been found using a widely available commercial bisulfite-based assay for distinguishing methylated from unmethylated cytosine. Cytosines analyzed for this invention were not limited to CpG islands or to specific genes but included cytosine loci outside of CpG islands and outside of genes. Cytosine loci associated with known genes are highlighted however extragenic loci also showed significant differences in methylation which are useful in distinguishing autism from normal cases. Multiple individual cytosine loci demonstrate highly significant differences in the degree of their methylation in autism versus normal cases (FDR q-values 1.0×10−3 to 1.0×10−35) see below.

Panels of known and identifiable cytosine loci throughout the genome whose methylation levels (expressed as percentages) are useful for distinguishing autism from normal cases and can be found in Tables 1-4.

In this example, 14 cases of autism diagnosed by neurologists and being been followed in a neurology clinic were compared to 10 normal controls. Demographic and clinical parameters were compared between the two groups (Tables 8-10).

TABLE 8
Comparison of Autism and Control Groups.

| | | Mean | | | Mean Age at |
|---|---|---|---|---|---|
| | | Maternal | Mean GA at | | Collection |
| | | Age in Years | Delivery in | Mean BW in | in Hours |
| Group | N | (SD) | Weeks (SD) | kg (SD) | (SD) |
| Case | 14 | 30.93 (8.10) | 39.21 (1.05) | 3.24 (0.39) | 32 (16.6) |
| Control | 10 | 32.0 (16.6) | 38.8 (1.23) | 3.52 (0.13) | 30.9 (8.1) |
| P-value (2 | — | 0.24 | 0.39 | 0.12 | 0.8 |
| tailed test) | | | | | |

TABLE 9
Newborn Gender.

| | | | | P-Value* |
|---|---|---|---|---|
| Gender | N (Total) | Case N (%) | Control N (%) | (Overall) |
| Male | 15 | 12 (85.7%) | 3 (30%) | 0.01 |
| Female | 9 | 2 (14.3%) | 7 (70%) | — |

*Fisher's Exact Test

TABLE 10
Maternal Race.

| | | | | P-Value* |
|---|---|---|---|---|
| Race | N (Total) | Case N (%) | Control N (%) | (Overall) |
| White | 14 | 11 (78.6%) | 3 (30%) | 0.035 |
| Black | 10 | 3 (21.4%) | 7 (70%) | — |

Highly significant differentiation in methylation levels were identified in cytosine loci when autism was compared to the normal group. The top 100 differentially methylated cytosine loci located within known genes and the associated genes are shown in Tables 5 and 7.

The cytosine loci were ranked based on FDR p-value significance level and area under the ROC curve of each locus. We found highly significant differences in cytosine methylation levels (similarly highly significant methylation differences were found in a large number of loci both intra- and extragenic in location however these are not presented in the tables). Different combinations of these methylation loci were used for the prediction of autism (Tables 1-4). Both complex models in which a relatively large number of marker loci and parsimonious models in which a smaller number of loci were used for autism prediction are considered. In each table the percentage contribution of each locus to discriminating autism from normal cases are provided. The inclusion of demographic and clinical characteristics such as maternal race, gestational age at delivery, newborn gender, birth weight and interval from birth to specimen collection did not contribute to autism prediction when considered in the predictive models (Tables 3 and 4). As shown in Table 6, multiple different biomarker combinations were very sensitive predictors of autism status.

The data shows a strong association between cytosine methylation status at a large number of cytosine sites throughout the genome using stringent False Discover Rate (FDR) using Benjamini-Hochberg test, analysis with q-values <0.05 and with many q-values as low as <1×10⁻³⁰, depending on particular cytosine locus being considered (Tables 1-4). Importance of methylated sites were ranked based on (low) FDR p-value and (high) AUC. A total of 14 cases of autism and 10 normal controls were evaluated.

The cytosine methylation markers reported enable population screening studies for the prediction and detection of autism based on cytosine methylation throughout the genome. They also permit improved understanding of the mechanism of development of autism. Understanding the mechanism is crucial to the identification of environmental factors including toxins, pharmaceutical agents, and recreational substances e.g. alcohol and narcotic drugs that contribute to the development of autism, and monitoring and mitigating the impact of such agents on autism development and severity. In addition, understanding the mechanism of development of autism can play an important role in development of specific therapies including pharmaceutical agents for autism treatment. Based on the above discussion, gene ontology analysis of cellular pathways involved in autism was performed (Table 11).

TABLE 11
Cellular pathways.

| | | | | Fold | |
|---|---|---|---|---|---|
| Category | ID | Pathway | P-value | Enrichment | FDR |
| GOTERM_CC_FAT | GO: 0044459 | plasma membrane | 6.56E−10 | 1.288033872 | 9.82E−07 |
| | | part | | | |
| GOTERM_CC_FAT | GO: 0005886 | plasma membrane | 1.35E−06 | 1.154598265 | 0.002024726 |
| GOTERM_CC_FAT | GO: 0045202 | synapse | 2.90E−06 | 1.620713951 | 0.004346224 |
| GOTERM_CC_FAT | GO: 0031012 | extracellular matrix | 1.88E−05 | 1.572935987 | 0.028194889 |
| GOTERM_CC_FAT | GO: 0005578 | proteinaceous | 6.90E−05 | 1.552800512 | 0.103228181 |
| | | extracellular matrix | | | |
| GOTERM_CC_FAT | GO: 0031226 | intrinsic to plasma | 4.42E−04 | 1.226904108 | 0.660518374 |
| | | membrane | | | |
| GOTERM_CC_FAT | GO: 0005901 | caveola | 4.45E−04 | 2.388923864 | 0.664168038 |
| GOTERM_CC_FAT | GO: 0030054 | cell junction | 5.18E−04 | 1.363157531 | 0.773145321 |
| GOTERM_CC_FAT | GO: 0005856 | cytoskeleton | 7.04E−04 | 1.202519395 | 1.049170132 |
| GOTERM_CC_FAT | GO: 0005887 | integral to plasma | 8.66E−04 | 1.21626409 | 1.28909368 |
| | | membrane | | | |
| GOTERM_CC_FAT | GO: 0009898 | internal side of | 0.0010998 | 1.448314934 | 1.634576305 |
| | | plasma membrane | | | |
| GOTERM_CC_FAT | GO: 0009986 | cell surface | 0.001598077 | 1.409074874 | 2.36690154 |
| GOTERM_CC_FAT | GO: 0044420 | extracellular matrix | 0.00242136 | 1.732319059 | 3.565755828 |
| | | part | | | |
| GOTERM_CC_FAT | GO: 0019717 | synaptosome | 0.003600736 | 1.846053859 | 5.259227243 |
| GOTERM_CC_FAT | GO: 0005581 | collagen | 0.004254465 | 2.428439898 | 6.185924151 |
| GOTERM_CC_FAT | GO: 0044456 | synapse part | 0.00519834 | 1.435194311 | 7.50900302 |
| GOTERM_CC_FAT | GO: 0015629 | actin cytoskeleton | 0.005579245 | 1.409703458 | 8.03798951 |
| GOTERM_CC_FAT | GO: 0044433 | cytoplasmic vesicle | 0.00570152 | 1.503415074 | 8.207200255 |
| | | part | | | |
| GOTERM_CC_FAT | GO: 0005884 | actin filament | 0.006343642 | 2.232524484 | 9.091046234 |
| GOTERM_CC_FAT | GO: 0000267 | cell fraction | 0.010043393 | 1.171184523 | 14.03078185 |
| GOTERM_CC_FAT | GO: 0031225 | anchored to | 0.0149852 | 1.396777494 | 20.23877552 |
| | | membrane | | | |
| GOTERM_CC_FAT | GO: 0044421 | extracellular region | 0.019058687 | 1.164600384 | 25.03869134 |
| | | part | | | |
| GOTERM_CC_FAT | GO: 0012506 | vesicle membrane | 0.019434305 | 1.472156637 | 25.46744234 |
| GOTERM_CC_FAT | GO: 0005626 | insoluble fraction | 0.019875422 | 1.17670348 | 25.96803535 |
| GOTERM_CC_FAT | GO: 0042734 | presynaptic | 0.024882943 | 2.254519799 | 31.43500872 |
| | | membrane | | | |
| GOTERM_CC_FAT | GO: 0030659 | cytoplasmic vesicle | 0.029219251 | 1.458139064 | 35.86237471 |
| | | membrane | | | |
| GOTERM_CC_FAT | GO: 0005624 | membrane fraction | 0.031562903 | 1.163766957 | 38.14271008 |
| GOTERM_CC_FAT | GO: 0045211 | postsynaptic | 0.033468218 | 1.452912759 | 39.94055046 |
| | | membrane | | | |
| GOTERM_CC_FAT | GO: 0043005 | neuron projection | 0.034042287 | 1.261740028 | 40.47260211 |
| GOTERM_CC_FAT | GO: 0030934 | anchoring collagen | 0.036088032 | 3.632281898 | 42.33306135 |

There was over representation of pathways related to plasma membrane, nerve synapse and proteinaceous extracellular matrix. These were statistically significant (FDR) when controlled for multiple comparisons.

The cytosines evaluated include those in CpG islands located in the promoter regions of the genes. Other areas targeted and measured include the so called CpG island ‘shores’ located up to 2000 base pairs distant from CpG islands and ‘shelves’ which is the designation for DNA regions flanking shores. Even more distant areas from the CpG islands so called “seas” were analyzed for cytosine methylation differences. Thus comprehensive and genome-wide analysis of cytosine methylation was performed. Although sites exhibited in tables were confined to those associated with known genes, the genome-wide analysis is described above.

Numerous genes are referenced herein. Exemplary sequences can be found in the accompanying sequence listing, as follows: BCL6 co-repressor (BCOR; SEQ ID NO: 1); long intergenic non-protein coding RNA 589 (C8orf75; SEQ ID NO: 2); voltage-sensitive chloride channel 1 (CLCN1; SEQ ID NO: 3); voltage-sensitive chloride channel 4 (CLCN4; SEQ ID NO: 4); disco-interacting protein 2 homolog C (D1 P2C; SEQ ID NO: 5); glycoprotein M6B (GPM6B; SEQ ID NO: 6); integrin, alpha X complement component 3 receptor 4 subunit (ITGAX; SEQ ID NO: 7); LOC284412 (SEQ ID NO: 8);

long intergenic non-protein coding RNA 620 (LOC285375; SEQ ID NO: 9); mastermind-like domain containing 1 (MAMLD1; SEQ ID NO: 10); MIR503 host gene (MGC16121; SEQ ID NO. 11); NADH dehydrogenase (ubiquinone) 1 alpha subcomplex, 10 (NDUFA10; SEQ ID NO: 12); phosphodiesterase 9A (PDE9A; SEQ ID NO: 13); phosphatidic acid phosphatase type 2 domain containing 1A (PPAPDC1A; SEQ ID NO: 14); protein tyrosine phosphatase, receptor type, N polypeptide 2 (PTPRN2; SEQ ID NO: 15); RAP1 GTPase activating protein 2 (RAP1GAP2; SEQ ID NO: 16); ribosomal protein S4, Y-linked 2 (RPS4Y2; SEQ ID NO: 17); tubulin alpha 3d (TUBA3D; SEQ ID NO: 18); and ubiquitin domain containing 1 (UBTD1; SEQ ID NO: 19). Numerous additional sequences can be found in publicly available databases known to one of ordinary skill in the art.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in the ability to predict and/or diagnose autism in a subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1-68. (canceled)

69. A method of predicting autism in a subject comprising:
obtaining a sample from the subject or from the subject's mother when the subject is at an embryonic or fetal stage of life;
assaying the sample to determine a percentage of methylated cytosine nucleotides at one or more of the following genes: BCL6 co-repressor (BCOR); long intergenic non-protein coding RNA 589 (C8orf75); chloride channel voltage-sensitive 1 (CLCN1); chloride channel voltage-sensitive 4 (CLCN4); disco-interacting protein 2 homolog C (D1P2C); glycoprotein M6B (GPM6B); integrin, alpha X complement component 3 receptor 4 subunit (ITGAX); LOC284412; long intergenic non-protein coding RNA 620 (LOC285375); mastermind-like domain containing 1 (MAMLD1); MIR503 host gene (MGC16121); NADH dehydrogenase (ubiquinone) 1 alpha subcomplex, 10 (NDUFA10); phosphodiesterase 9A (PDE9A); phosphatidic acid phosphatase type 2 domain containing 1A (PPAPDC1A); protein tyrosine phosphatase, receptor type, N polypeptide 2 (PTPRN2); RAP1 GTPase activating protein 2 (RAP1GAP2); ribosomal protein S4, Y-linked 2 (RPS4Y2); tubulin, alpha 3d (TUBA3D); or ubiquitin domain containing 1 (UBTD1);
obtaining a value based on the assay;
comparing the value to a reference level; and
predicting that the subject has autism based on the methylation of cytosine nucleotides as demonstrated by the value and the reference level.

70. The method of claim 69, wherein assaying the sample comprises determining a percentage of methylated cytosine nucleotides at one or more of the following genes: BCOR, C8orf75, CLCN1. CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, or UBTD1.

71. The method of claim 69, wherein assaying the sample comprises determining percentage of methylated cytosine nucleotides at the following genes: BCOR, PTPRN2, TUBA3D, PDE9A, and LOC284412.

72. The method of claim 69, wherein assaying the sample comprises determining percentage of methylated cytosine nucleotides at the following genes: GPM6B, NDUFA10, PDE9A, and LOC284412.

73. The method of claim 69, wherein assaying the sample comprises determining percentage of methylated cytosine nucleotides at the following genes: BCOR, UBTD1, LOC285375, RPS4Y2, PPAPDC1A, ITGAX, D1P2C, MGC16121, PTPRN2, and CLCN4.

74. The method of claim 69, wherein assaying the sample comprises determining percentage of methylated cytosine nucleotides at the following genes: RAP1GAP2, UBTD1, MAMLD1, and C8orf75.

75. The method of claim 69, wherein the sample is one or more of the following samples: a tissue sample, a cell sample, a body fluid sample, a whole blood sample, a serum sample, a plasma sample, a saliva sample, a genital secretion sample, a sputum sample, a urine sample, a CSF sample, an amniotic fluid sample, a tear sample, a buccal sample, or a breath condensate sample.

76. The method of claim 75, wherein the sample is maternal blood, amniotic fluid, or placental tissue.

77. The method of claim 69, wherein the value is a weighted score.

78. The method of claim 69, wherein the sample is obtained while the subject is at an embryonic stage, a fetal stage, a neonatal stage, an infant stage, a childhood stage, an adolescent stage, or an adulthood stage.

79. A method of diagnosing autism in a subject comprising:
obtaining a sample from the subject or the subject's mother when the subject is at an embryonic or fetal stage of life;
assaying the sample to determine a percentage of methylated cytosine nucleotides at one or more of the following genes: BCL6 co-repressor (BCOR); long intergenic non-protein coding RNA 589 (C8orf75); chloride channel voltage-sensitive 1 (CLCN1); chloride channel voltage-sensitive 4 (CLCN4); disco-interacting protein 2 homolog C (D1P2C); glycoprotein M6B (GPM6B); integrin, alpha X complement component 3 receptor 4 subunit (ITGAX); LOC284412; long intergenic non-protein coding RNA 620 (LOC285375); mastermind-like domain containing 1 (MAMLD1); MIR503 host gene (MGC16121); NADH dehydrogenase (ubiquinone) 1 alpha subcomplex, 10 (NDUFA10); phosphodiesterase 9A (PDE9A); phosphatidic acid phosphatase type 2 domain containing 1A (PPAPDC1A); protein tyrosine phosphatase, receptor type, N polypeptide 2 (PTPRN2); RAP1 GTPase activating protein 2 (RAP1GAP2); ribosomal protein S4, Y-linked 2 (RPS4Y2); tubulin, alpha 3d (TUBA3D); or ubiquitin domain containing 1 (UBTD1);
obtaining a value based on the assay;
comparing the value to a reference level; and
diagnosing the subject with autism based on the methylation of cytosine nucleotides as demonstrated by the value and the reference level.

80. The method of claim 79, wherein assaying the sample comprises determining a percentage of methylated cytosine nucleotides at one or more of the following genes: BCOR, C8orf75, CLCN1. CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, or UBTD1.

81. The method of claim 79, wherein assaying the sample comprises determining percentage of methylated cytosine nucleotides at the following genes: BCOR, PTPRN2, TUBA3D, PDE9A, and LOC284412.

82. The method of claim 79, wherein assaying the sample comprises determining percentage of methylated cytosine nucleotides at the following genes: GPM6B, NDUFA10, PDE9A, and LOC284412.

83. The method of claim 79, wherein assaying the sample comprises determining percentage of methylated cytosine nucleotides at the following genes: BCOR, UBTD1, LOC285375, RPS4Y2, PPAPDC1A, ITGAX, D1P2C, MGC16121, PTPRN2, and CLCN4.

84. The method of claim 79, wherein assaying the sample comprises determining percentage of methylated cytosine nucleotides at the following genes: RAP1GAP2, UBTD1, MAMLD1, and C8orf75.

85. The method of claim 79, wherein the sample is one or more of the following samples: a tissue sample, a cell sample, a body fluid sample, a whole blood sample, a serum sample, a plasma sample, a saliva sample, a genital secretion sample, a sputum sample, a urine sample, a CSF sample, an amniotic fluid sample, a tear sample, a buccal sample, or a breath condensate sample.

86. The method of claim 85, wherein the sample is maternal blood, amniotic fluid, or placental tissue.

87. The method of claim 79, wherein the value is a weighted score.

88. The method of claim 79, wherein the sample is obtained while the subject is at an embryonic stage, a fetal stage, a neonatal stage, an infant stage, a childhood stage, an adolescent stage, or an adulthood stage.

89. A kit for diagnosing or predicting autism in a subject comprising one or more probes and/or one or more microarrays designed to identify and/or assay methylation of cytosines at one or more genes in a sample from a subject or from a subject's mother when the subject is at an embryonic or fetal stage of life, wherein the one or more of the genes is one or more of the following genes: BCL6 co-repressor (BCOR); long intergenic non-protein coding RNA 589 (C8orf75); chloride channel voltage-sensitive 1 (CLCN1); chloride channel voltage-sensitive 4 (CLCN4); disco-interacting protein 2 homolog C (D1P2C); glycoprotein M6B (GPM6B); integrin, alpha X complement component 3 receptor 4 subunit (ITGAX); LOC284412; long intergenic non-protein coding RNA 620 (LOC285375); mastermind-like domain containing 1 (MAMLD1); MIR503 host gene (MGC16121); NADH dehydrogenase (ubiquinone) 1 alpha subcomplex, 10 (NDUFA10); phosphodiesterase 9A (PDE9A); phosphatidic acid phosphatase type 2 domain containing 1A (PPAPDC1A); protein tyrosine phosphatase, receptor type, N polypeptide 2 (PTPRN2); RAP1 GTPase activating protein 2 (RAP1GAP2); ribosomal protein S4, Y-linked 2 (RPS4Y2); tubulin, alpha 3d (TUBA3D); or ubiquitin domain containing 1 (UBTD1).

90. The kit of claim 89, where the one or more microarrays comprise one or more probes that identify and/or assay for methylation of cytosines at
(a) one or more of the following genes: BCOR, C8orf75, CLCN1, CLCN4, D1P2C, GPM6B, ITGAX, LOC284412, LOC285375, MAMLD1, MGC16121, NDUFA10, PDE9A, PPAPDC1A, PTPRN2, RAP1GAP2, RPS4Y2, TUBA3D, or UBTD1;
(b) the following genes: BCOR, PTPRN2, TUBA3D, PDE9A, and LOC284412;
(c) the following genes: GPM6B, NDUFA10, PDE9A, and LOC284412;
(d) the following genes: BCOR, UBTD1, LOC285375, RPS4Y2, PPAPDC1A, ITGAX, D1P2C, MGC16121, PTPRN2, and CLCN4; or
(e) the following genes: RAP1GAP2, UBTD1, MAMLD1, and C8orf75.

91. The method of claim 69, wherein the method further comprises obtaining cell free DNA from the sample.

92. The method of claim 79, wherein the method further comprises obtaining cell free DNA vrom the sample.
